(19) United States
(12) Patent Application Publication
(10) Pub. No.: US 2024/0408033 A1
(43) Pub. Date: Dec. 12, 2024

(54) CELL-DERIVED NANOVESICLES FOR IN VIVO TRANSPORT AND DELIVERY OF THERAPEUTIC MATERIALS

(71) Applicant: The Board of Trustees of the Leland Stanford Junior University, Stanford, CA (US)

(72) Inventors: Ramasamy Paulmurugan, Stanford, CA (US); Tarik Massoud, Stanford, CA (US); Uday Kumar Sukumar, Stanford, CA (US)

(21) Appl. No.: 18/700,890

(22) PCT Filed: Oct. 14, 2022
(86) PCT No.: PCT/US22/46708

§ 371 Date: Apr. 12, 2024

Related U.S. Application Data
(60) Provisional application No. 63/256,089, filed on Oct. 15, 2021.

(30) Foreign Application Priority Data
Oct. 15, 2021 (US) 63256089

(51) Int. Cl. A61K 9/51; A61K 31/138; A61K 31/337; A61K 31/44; A61K 31/495; A61K 31/573; A61K 31/704; A61K 31/7068; A61K 47/64; A61K 47/68; A61K 47/69; C12N 15/113

(52) U.S. Cl. CPC A61K 9/5176; A61K 9/5192; A61K 31/138; A61K 31/337; A61K 31/44; A61K 31/495; A61K 31/573; A61K 31/704; A61K 31/7068; A61K 47/64; A61K 47/6849; A61K 47/6901; C12N 15/113; C12N 2310/14; C12N 2310/141

(57) ABSTRACT

The invention provides compositions comprising a plurality of nanovesicles formed from cell-derived extracellular vesicles (“EV”) and loaded with one or more imaging agents or therapeutic agents, and related compositions and methods for delivery of the therapeutic agents to a target cell or tissue and for related methods of treatment and imaging.

### CROSS-REFERENCE TO RELATED APPLICATIONS

This application claims the benefit of priority of U.S. provisional application No. 63/256,089 filed Oct. 15, 2021, the contents of which are hereby incorporated by reference in their entirety.

### STATEMENT AS TO FEDERALLY SPONSORED RESEARCH

This invention was made with Government support under contracts CA209888 and EB022298 awarded by the National Institutes of Health. The Government has certain rights in the invention.

### BACKGROUND

Synthetic naked therapeutic RNAs, such as miRNAs, are sensitive to nucleases and effective delivery agents are required to protect them in biological environments. Synthetic polymers, virus-based vectors, and lipid-based vectors have been studied as delivery vehicles for nucleic acids, including RNAs and miRNAs. However, biological toxicity, immunogenicity, and low effectiveness hinder successful clinical translation of these carriers. In comparison, extracellular vesicles (EVs) derived from various bacteria or mammalian cells have higher delivery efficiency and better biocompatibility. However, EV-based delivery systems also suffer from a number of difficulties which have hindered their clinical use, including the failure to achieve therapeutic drug doses at the target site. The present invention addresses the need for more efficient loading of therapeutic agents in EV-based delivery systems to increase drug delivery to the target site.

### BRIEF SUMMARY

The invention provides nanovesicle compositions, including pharmaceutical compositions, and related products and methods, the nanovesicle compositions comprising a plurality of nanovesicles encapsulating one or more therapeutic agents, wherein the nanovesicles have a mean particle diameter of from about 90-150 nanometers, from about 100-200 nanometers, from about 200-300 nanometers, from about 300-400 nanometers, or from about 400-500 nanometers, and wherein the nanovesicles comprise cell membranes of a source cell. In embodiments, the invention provides a composition comprising a plurality of nanovesicles, each nanovesicle comprising one or more therapeutic agents, wherein the nanovesicles have a mean particle diameter of from about 90-150 nanometers.

In embodiments, the cell-derived extracellular vesicles are derived from a cell selected from the group consisting of stem cells, platelets, erythrocytes, lymphocytes, dendritic cells, monocytes, and tumor cells; optionally wherein the stem cells are neural stem cells; and further optionally wherein the tumor cells are breast cancer cells.

In embodiments, the nanovesicles comprise a recombinant membrane associated protein, antibody, affibody, or peptide on the external surface of the nanovesicles, optionally wherein the membrane associated protein binds to a ligand on the surface of a target cell. In embodiments, the recombinant membrane associated protein is a CXCR4 receptor. In embodiments, the antibody is selected from an anti-transferrin receptor antibody, an anti-uPAR antibody (alternatively an anti-CD87 antibody), an anti-HER2 antibody, or a B7-H3 antibody. In embodiments, the affibody is selected from an anti-transferrin receptor affibody, an anti-uPAR affibody, an anti-HER2-affibody, or a B7-H3 affibody. In embodiments, the peptide is selected is from a T7-peptide, a urokinase plasminogen activator (uPA) peptide, or an SP94 peptide.

In embodiments, the one or more therapeutic agents is a nucleic acid or a small molecule therapeutic agent. In this context, a small molecule therapeutic agent refers to an organic molecule having a low molecular weight, generally below about 1000 daltons or below about 900 daltons. In embodiments, the one or more therapeutic agents is a small molecule selected from one or more of doxorubicin, dexamethasone, gemcitabine, paclitaxel, sorafenib, tamoxifen, 4-hydroxy tamoxifen, or temozolomide. In embodiments, the one or more therapeutic agents is an RNA or DNA. In embodiments, the RNA is a small interfering RNA (siRNA) or a microRNA (miRNA). In embodiments, the RNA is a microRNA. In embodiments, the microRNA is miR-21, miR-10b, miR-100, miR-122, and mixtures thereof. In embodiments, the RNA encodes one or more antigenic or immunostimulatory proteins, for example in the context of an RNA vaccine composition.

The disclosure also provides methods for loading a therapeutic agent into a plurality of cell-derived microvesicles, the method comprising subjecting a mixture of the therapeutic agent and microvesicles to microfluidic processing to produce loaded microfluidic processed extracellular vesicles (mpEVs) which may optionally be further subjected to ultracentrifugation in order to further purify and concentrate the mpEVs. The mpEVs are of uniform size, preferably having a mean particle diameter of from about 90-150 nanometers, and may also be referred to herein as “nanovesicles”. In embodiments, the cells are stem cells. In embodiments, the cells are neural cells, such as neural stem cells. In embodiments, the cells are dendritic cells. In embodiments, the cells are breast cancer cells. In embodiments, the microfluidic processing step comprises subjecting the mixture to a pressure of about 20,000-40,000 psi. In embodiments, the microvesicles are obtained from cells expressing a recombinant membrane associated protein characterized in that the recombinant protein binds to a ligand on the surface of a target cell.

The disclosure also provides a plurality of nanovesicles loaded with a therapeutic agent, such as an miRNA and/or small organic molecule, produced by the methods described here.

The disclosure also provides methods of treating a disease or disorder, the method comprising administering to a subject in need of such treatment a composition comprising a plurality of nanovesicles as described herein, wherein the one or more therapeutic agents loaded in the nanovesicles is indicated for treatment of the disease or disorder. In embodiments, the disease or disorder is a cancer. In embodiments, the cancer is a glioblastoma, a breast cancer, hepatocellular carcinoma, or a lung cancer. In embodiments, the nanovesicles comprise a recombinant membrane associated protein, antibody, affibody, or peptide on the external surface of the nanovesicles, optionally wherein the membrane associated protein binds to a ligand on the surface of a target cell. In embodiments, the recombinant membrane associated protein is a CXCR4 receptor. In embodiments, the antibody is selected from an anti-transferrin receptor antibody, an anti-uPAR antibody (also referred to as an anti-CD87 antibody), an anti-HER2 antibody, or a B7-H3 antibody. In embodiments, the affibody is selected from an anti-transferrin receptor affibody, an anti-uPAR affibody, an anti-HER2-affibody, or a B7-H3 affibody. In embodiments, the peptide is selected is from a T7-peptide, a urokinase plasminogen activator (uPA) peptide, or an SP94 peptide.

In embodiments, the cancer is a glioblastoma and the one or more therapeutic agents loaded in the nanovesicles is a microRNA or a small molecule therapeutic agent selected from one or more of temozolomide, doxorubicin, or dexamethasone. In embodiments, the nanovesicles comprise from about 500-800 copies of nucleic acids consisting of a mixture of anti-miRNA-21 and miRNA-100. In embodiments, the nanovesicles comprise a recombinant membrane associated protein, antibody, affibody, or peptide on the external surface of the nanovesicles. In embodiments, the recombinant membrane associated protein is a CXCR4 receptor. In embodiments, the antibody is an anti-transferrin receptor antibody. In embodiments, the affibody is an anti-transferrin receptor affibody. In embodiments, the peptide is a T7-peptide. In embodiments, the method further comprises administering temozolomide, doxorubicin, dexamethasone, or any combination thereof to the subject.

In embodiments, the cancer is breast cancer and the one or more therapeutic agents is a nucleic acid or a small molecule therapeutic agent selected from one or more of tamoxifen, 4-hydroxy tamoxifen, paclitaxel, and doxorubicin. In embodiments, the nanovesicles comprise a recombinant membrane associated protein, antibody, affibody, or peptide on the external surface of the nanovesicles. In embodiments, the antibody is an anti-uPAR antibody, an anti-HER2 antibody, or a B7-H3 antibody. In embodiments, the affibody is an anti-uPAR affibody, an anti-HER2-affibody, or a B7-H3 affibody. In embodiments, the peptide is a urokinase plasminogen activator (uPA) peptide. In embodiments, the method further comprises administering tamoxifen, 4-hydroxy tamoxifen, paclitaxel, doxorubicin, or any combination thereof, to the subject.

In an embodiment, the invention provides a pharmaceutical composition for treating breast cancer, the composition comprising a plurality of nanovesicles each comprising one or more therapeutic agents, wherein the nanovesicles comprise a membrane expressed anti-uPAR antibody, anti-uPAR affibody, or urokinase plasminogen activator (uPA) peptide. In embodiments, the one or more therapeutic agents includes antisense-miRNA-21, also referred to as anti-miRNA-21. In embodiments, the nanovesicles further comprise poly(lactic-co-glycolic acid) (PLGA). In embodiments, the nanovesicles form a membrane coating around a PLGA particle comprising the one or more therapeutic agents, optionally wherein the one or more therapeutic agents includes antisense-miRNA-21.

In embodiments, the cancer is hepatocellular carcinoma and the nanovesicles comprise a peptide on the external surface of the nanovesicles, optionally wherein the peptide is an SP94 peptide. In embodiments, the one or more therapeutic agents is sorafenib, gemcitabine, or doxorubicin. In embodiments, the method further comprises administering sorafenib, gemcitabine, doxorubicin, or any combination thereof to the subject. In embodiments, the nanovesicles further comprise poly(lactic-co-glycolic acid) (PLGA). In embodiments, the nanovesicles form a membrane coating around a PLGA particle comprising the one or more therapeutic agents, optionally wherein the one or more therapeutic agents includes orafenib, gemcitabine, or doxorubicin.

The disclosure also provides a composition comprising a plurality of nanovesicles, each comprising one or more therapeutic agents, wherein the nanovesicles of the plurality have a mean particle diameter of from about 90-150 nanometers and wherein the nanovesicles further comprise a coating. In embodiments, the coating is a metallic coating, optionally wherein the metallic coating is gold, iron, or silver. In embodiments, the coating is a polymeric coating, optionally wherein the polymeric coating is poly(lactic-co-glycolic acid) (PLGA), PLGA-poly(ethylene glycol) (PEG), or chitosan. In embodiments, the nanovesicles are obtained from dendritic cell membranes. In embodiments, the composition is a vaccine composition.

The disclosure also provides a composition comprising a plurality of nanovesicles, each comprising one or more therapeutic agents, wherein the nanovesicles comprise a cell-derived membrane encapsulating a polymer-based nanoparticle, such as PLGA-based nanoparticle, comprising one or more therapeutic agents or imaging agents, or both.

### BRIEF DESCRIPTION OF THE DRAWINGS

FIG. 1A is a schematic illustrating release of extracellular vesicles (EVs) by auto exocytosis from neural stem cells (NSC), including NSCs engineered to overexpress CXCR4 surface receptors.

FIG. 1B is a schematic illustrating collection of EVs followed by labeling with ICG NIR dye. Loading of microRNAs, miR100 and Cy5-AmiR21 is performed by subjecting the EVs and miRNAs to microfluidic processing to provide ICG-labeled miRNA loaded mpEVs.

FIG. 1C is a schematic illustrating an mpEV expressing cell surface CXCR4 and ICG label loaded with miRNAs, miRNA-100 (“miR 100”) and Cy5-labeled anti-miRNA-21 (“AmiR21”).

FIG. 1D is a schematic illustrating intranasal administration of mpEVs in mice bearing orthotopic GBMs.

FIG. 1E shows theranostic imaging in GBM of living mice. mpEVs were monitored using multimodal imaging including optical bioluminescence for viable tumor load determination, ICG-based fluorescence for mpEV load determination, and photoacoustic imaging. Tumor volume was determined using magnetic resonance imaging (MRI).

FIG. 2A shows flow cytometric (FACS) analysis of NSCs engineered to overexpress CXCR4 receptors and detected using anti-CXCR4 antibody (filled curve), compared with isotype control antibody labeled cells and unstained control cells (overlapping traces to the left of filled curve).

FIG. 2B shows western blot analysis of the expression of CXCR4 proteins in engineered and control NSCs: Chemiluminescence images of western bands (top) and the quantitative graph (bottom).

FIG. 2C shows Cy5-miRNA loading efficiency in EVs obtained by transfection versus microfluidic processing as measured using FACS analysis detecting Cy5-miR fluorescence. Left-most trace is empty EV; middle overlapping traces are control EV and CXCR4 EV both transfected with anti-miRNA-21 (“AmiR21”); overlapping right-most traces are control and CXCR4 mpEV both loaded with AmiR21 by microfluidic processing.

FIG. 2D shows quantification of anti-miRNA-21 (“AmiR 21”) and miRNA-100 (“miR 100”) levels in mpEVs obtained by transfection versus microfluidic processing using quantitative RT-PCR. Starting from left of graft, first bar is control EV, next two bars are control EV and CXCR4 EV, both transfected with either AmiR 21 (dark bars) or miR 100 (light bars); last three bars are control mpEV, and control mpEV and CXCR4 mpEV both loaded with either AmiR 21 (dark bars) or miR 100 (light bars) by microfluidic processing.

FIG. 3 illustrates characterization of NSC mpEVs labeled using ICG NIR fluorescent dye and produced using microfluidic processing. ICG-NIR dye labeling efficiency of control and CXCR4-NSC mpEVs was evaluated by (i) EV proteins resolved in SDS-PAGE stained by Coomassie blue, (ii) ICG-fluorescence imaging, (iii) Cy5-miRNA fluorescence imaging, and (iv) intact mpEVs imaged for loaded Cy5-miRNA and ICG by fluorescence imaging.

FIG. 4A shows FACS analysis of miRNA-loaded mpEVs uptake in U87MG GBM cells at different time point (24 h and 48 h) with different TMZ doses (0 μM, 250 μM, 500 μM) to evaluate variations in responses to TMZ treatment.

FIG. 4B shows quantitative RT-PCR analysis of U87MG GBM cells treated with miRNAs-loaded control mpEVs and CXCR4-NSC mpEVs carrying different miRNA concentrations (25 pmol and 50 pmol) for the delivered intracellular anti-miRNA-21 levels.

FIG. 4C shows quantitative RT-PCR analysis of U87MG GBM cells treated with miRNA-loaded control and CXCR4-NSC-mpEVs carrying different miRNA concentrations (25 pmol and 50 pmol) for therapeutic silencing effects on endogenous miRNA-21 expression using the delivered anti-miRNA-21.

FIG. 4D shows quantitative RT-PCR analysis of U87MG GBM cells treated with miRNA-loaded control and CXCR4-NSC-mp EVs carrying different miRNA concentrations (25 pmol and 50 pmol) for the delivered intracellular miRNA-100 levels.

FIG. 5 shows data of an evaluation of the chemosensitizing effects of miRNA-loaded NSC-mp EVs delivered to U87MG glioma cells and co-treated with temozolomide (TMZ). Plots depict propidium iodide (PI) staining-based apoptosis FACS analysis of U87MG GBM cells treated with different combinations of NSC-mpEVs (with and without miRNA loading) plus TMZ.

FIG. 6A is a schematic outline of the experimental design and timelines adopted for in vivo treatments and imaging of intranasal delivered control and CXCR4-NSC-mpEVs loaded with miRNAs and co-treated with systemic TMZ.

FIG. 6B shows body weight over time for animals from different treatment groups (n=4) to monitor the impact of treatments on animal health.

FIG. 6C shows survival curves of animals from different treatment groups (n=4). Note although 5 animals were included in each group some animals were used for periodic histological evaluations. From left, the groups are untreated (21 days); control mpEV+miRNAs (24 days); CXCR4 mpEV+miRNAs (23 days); control mpEV+TMZ (28 days); control mpEV+miRNAs+TMZ (38 days); and CXCR4 mpEV+miRNAs+TMZ (48 days).

FIG. 7A shows biodistribution of intranasal delivered control (dark bars) and CXCR4-NSC-mpEVs (light bars) loaded with anti-miRNA-21 (“AmiR 21”) in major organs by qRT-PCR for delivered Anti-miR-21 and miR-100 in brain, lung, heart, liver, spleen and kidneys.

FIG. 7B shows biodistribution of intranasal delivered control (dark bars) and CXCR4-NSC-mpEVs (light bars) loaded with miRNA-100 (“miR 100”) in major organs by qRT-PCR for delivered Anti-miR-21 and miR-100 in brain, lung, heart, liver, spleen and kidneys.

FIG. 8A shows data of ex vivo imaging of brain harvested from mice after intranasal administration of ICG labeled mpEVs at different time points (30 min, 2 h, 12 h, 24 h) and their intracranial distribution with respect to the tumors after a single intranasal dose. Two animals were allocated for each time point.

FIG. 8B shows a schematic illustration of a sagittal view of the brain (top) and a bar chart (bottom) showing quantitative estimation of delivered AmiR-21 in fore brain (F), mid brain (M) and rear/hind brain (R).

FIG. 8C is a bar chart showing quantitative estimation of delivered AmiR-21 in heart, lung, liver, spleen, kidney, and brain at 30 min, 2 h, 12 h, 24 h (from left to right in each tissue group).

FIG. 9 is a graph showing an anti-miR-21 standard plot and the corresponding fitting curve derived by Tagman RT-PCR amplification of anti-miR21 using known copy numbers.

FIG. 10A-10D shows NTA analysis of control and CXCR4-EVs before and after microfluidic processing depicting their concentration and size distribution. FIG. 10E-10F show amounts of anti-miR-21 and miR-100 loaded in EVs and microfluidically processed mpEVs as determined by quantitative RT-PCR. FIGS. 10G and 10H show the amplification curves.

FIG. 11A-D illustrate data showing RNase protection assays to measure the loaded microRNA in mpEVs. FIGS. 11A and 11C shows surface zeta potential measurement of empty mpEVs and microRNA loaded mpEVs treated with or without RNase for the indicated times;

FIG. 11B shows quantitative estimation of loaded microRNAs in respective treatment conditions measured by TaqMan qRT-PCR; FIG. 11D shows RNase concentration dependent degradation of microRNAs loaded in mpEVs measured by qRT-PCR.

FIG. 12A-B are time dependent release profiles of AmiR21 microRNAs release from mpEVs by RT-PCR quantitation (FIG. 12A); and stability of ICG conjugated to mpEVs with respect to time (FIG. 12B).

FIG. 13 show an evaluation of the chemosensitizing effects of miRNA-loaded mpEVs delivered to GL-26 glioma cells and co-treated with temozolomide (TMZ). PI staining-based FACS analysis was utilized to measure apoptosis in GL-26 GBM cells treated with different combinations of mpEVs (with and without miRNA loading) plus TMZ (250 μM and 500 μM).

FIG. 14A-B shows a therapeutic evaluation of intranasally delivered control and CXCR4-NSC mpEVs loaded with miRNAs and co-treated with systemic TMZ. Tumor volume was assessed by MR imaging. In panel A, shown are T2- and T1-weighted MR images of mice brains in all treatment groups at different time points (D4, D11, D19, and D25) (n=5). The tumors were outlined by dashed borders in red. In panel B, shown are photoacoustic images utilized to evaluate accumulation of ICG-labeled NSC mpEVs.

FIG. 15 shows Kaplan Meier survival curves for different treatment conditions (n=3) in a therapeutic evaluation of intranasally delivered control-mpEVs, control microRNA loaded mpEVs, microRNA loaded CXCR4-mpEVs, and their respective combinations with TMZ treatment.

FIG. 16 shows paracellullar or transcellular transport of ICG labeled (red) EVs across olfactory mucosal membrane.

FIG. 17 shows time dependent (30 min, 2 h, 12 h, 24 h) transport of intranasally administered ICG labeled mpEVs through trigeminal and olfactory nerves into the brain and subsequent recruitment to U87MG tumors by intracranial migration through SDF-1α-CXCR4 axis.

FIG. 18 shows sagittal head sections of mice intranasally administered with ICG labeled mpEVs stained using Alexa Fluor 488@-phalloidin for actin filaments and Hoechst 33258 for nucleus by confocal microscopy. Respective H&E and three channel imaging of sagittal sections of animal heads with intranasal treatment using ICG labeled CXCR4-NSC-mpEVs showing the intracranial transport of EVs through the nose-to-brain pathways. The sections were stained with Alexa Fluor 488® phalloidin F-actin probe and Hoechst 33342 (blue), and the images were overlaid with signal from the ICG channel to depict the distribution and migration of EVs (red) in the nasal cavity and trigeminal route, and correlated with corresponding H&E-stained sections for pathological changes.

FIG. 19A-19P show showing in silico analysis based on data from The Cancer Genome Atlas (TCGA). Relative expression of (FIG. 19A) miRNA-21 and (FIG. 19B) miRNA-100 in different GBM subtypes and the adjacent normal brain tissue. The expression for each miRNA was normalized to normal brain tissue expressions. Both miRNAs were significantly upregulated in tumor compared to the adjacent normal tissue. Relative expression of (FIG. 19C) miRNA-21 and (FIG. 19D) miRNA-100 in low grade glioma (LGG). The expression for each miRNA was normalized to grade II expressions. **** shows P<0.0001. n.s. is a non-significant P-value. Therapeutic target genes of miRNA-21 and miRNA-100 were downregulated in some of the GBM subtypes based on the TCGA data. MiRNA-21 correlation with (FIG. 19E) PDCD4, and (FIG. 19F) PTEN in GBM subtypes. There was a clear negative correlation between the expression of miRNA-21 and PDCD4 as well as PTEN in proneural subtype GBM. MiR-100 correlation with (FIG. 19G) PLK1, and (FIG. 19H) CDKN1A in GBM subtypes. There was a negative correlation between miRNA-100 and PLK1 in proneural, and miRNA-100 and CDKN1A in classical, mesenchymal, and neural GBM subtypes. In graphs (FIG. 19E-H) the symbols show the data points and the matched color lines show the linear regression lines for each subtype: classical, green; mesenchymal, black, neural, red; and proneural, blue. GBM survival analysis of (FIG. 19I) miRNA-21 and (FIG. 19J) miRNA-100. Generally, the survival time for GBM patients is low (<10 months), and thus it is hard to see a significant difference between the survival of low and high expression levels. In (FIG. 19I) and (FIG. 19J) patients with lower expression of miRNAs had a slightly better survival. LGG survival analysis of (FIG. 19K) miRNA-21, and (FIG. 19L) miRNA-100. We observed significantly longer survival periods for patients in low expression groups for both miRNAs. Survival analysis in GBMs for (FIG. 19M) PLK1, and (FIG. 19N) PTEN. Survival analysis in LGG for (FIG. 19O) PLK1, and (FIG. 19P) PTEN. Patients with lower expression of PLK1 had a better survival, and those with higher expression of PTEN also showed better survival. The high and low expression groups correspond to the top and bottom 33% of patients in (FIG. 19I-19P).

### DETAILED DESCRIPTION

The disclosure provides compositions including pharmaceutical compositions comprising cell-derived nanovesicles and related nanoparticles encapsulating one or more therapeutic agents, including nucleic acid-based therapeutic agents, small organic molecule based therapeutic agents, and/or medical imaging agents, and related compositions and methods. The nanovesicles are formed from cell-derived extracellular vesicles (“EV”) of a source cell and comprise a phospholipid bilayer membrane of the source cell. In embodiments, the nanovesicles form nanoparticles encapsulating an interior space containing one or more therapeutic agents and/or imaging agents. In embodiments, the nanovesicles encapsulate a polymer-based nanoparticle, such as a poly(lactic-co-glycolic acid) (PLGA) nanoparticle, which comprises an interior space containing the one or more therapeutic agents.

Any cell can be utilized as a source cell for cell-derived EVs. Preferred cell types include stem cells, such as neural stem cells (NSC), blood and lymphatic cells, such as platelets, erythrocytes, lymphocytes, dendritic cells, and monocytes, or tumor cells, including for example breast cancer cells. The particular cell type may be selected to confer one or more source cell properties to the nanovesicles, including for example prolonged circulation time, immune system evasion, tumor targeting, and/or the ability to cross the blood brain barrier.

The nanovesicles described here are advantageously of substantially uniform size as determined by a narrow range in their mean particle diameter. In embodiments, the nanovesicles have a mean particle diameter in a range of from about 50-100 nanometers. In embodiments, the nanovesicles have a mean particle diameter of from about 90-150 nanometers, from about 100-200 nanometers, from about 200-300 nanometers, from about 300-400 nanometers, from about 400-500 nanometers, from about 500-600 nanometers, or from about 600-750 nanometers.

In embodiments, the disclosure provides an efficient microvesicle-based platform for targeted cell delivery of therapeutic and/or imaging agents, including nucleic acids and small molecule therapeutic agents and imaging agents. The disclosure provides a proof of concept example of this technology in the form of neural stem cell (NSC)-derived extracellular vesicles (EVs) adapted for delivery of a mixture of miRNA-based therapeutic agents to glioblastoma in a mouse model system.

Cell-derived extracellular vesicles, or “EVs”, typically will include a mixture of exosomes (30-100 nm) and microvesicles (MVs, 100-1000 nm). EVs are naturally secreted cell vesicles in the nanometer to micrometer size range, formed by outward budding of plasma membrane domains. Cells generally produce many more MVs compared to exosomes. Accordingly, the extracellular vesicles (EVs) described here consist primarily of MVs and the terms “microvesicle” and “EV” are used interchangeably herein. In embodiments, the source cells of the EVs are engineered to recombinantly express one or more membrane associated proteins, antibodies, affibodies, or peptides.

The disclosure also provides methods for high efficiency loading of therapeutic agents into EVs by processing the EVs and therapeutic agents together in a microfluidics system to produce loaded nanovesicles of uniform size containing high amounts of therapeutic agent, for example from 500-800 copies of a nucleic acid based therapeutic agent. The nanovesicles produced by these methods have a mean particle diameter of from about 90-150 nanometers (nm). The loaded nanovesicles containing therapeutic agent and having a uniform particle size may also be referred to herein as “microfluidically processed extracellular vesicles, or “mpEVs”.

In embodiments, the disclosure provides compositions comprising a plurality of nanovesicles, each comprising from about 500-800 copies of a therapeutic agent such as a nucleic acid or small molecule, wherein the nanovesicles of the plurality have a mean particle diameter of from about 90-150 nm. In embodiments, the nanovesicles are derived from a cell expressing a recombinant membrane associated protein, wherein the membrane associated protein targets the nanovesicles to a particular target cell by binding to a ligand on the target cell surface. Accordingly, in embodiments an mpEV of the invention comprises a recombinant membrane associated protein expressed on the external surface of the nanovesicles. In embodiments, the recombinant protein is a C-X-C chemokine receptor type 4 (CXCR4) receptor. CXCR4 is an alpha-chemokine receptor specific for stromal-derived-factor-1 (SDF-1, also called CXCL12), a molecule that is over-expressed in cancer cells, including GBM, breast cancer, and lung cancer.

In embodiments, the disclosure also provides methods of treating a disease or disorder by administering to a subject in need of therapy a composition comprising a plurality of nanovesicles, each comprising from about 500-800 copies of a therapeutic agent suitable for treating the disease or disorder, such as a nucleic acid or small molecule, wherein the nanovesicles of the plurality have a mean particle diameter of from about 90-150 nm. In embodiments, the mpEVs are used in a method of treating cancer and the mpEVs express a CXCR4 receptor on the external microvesicle surface. In embodiments, the cancer is CXCL12 expressing cancer, such as glioblastoma, breast cancer, or lung cancer. In embodiments, the mpEVs are derived from neural stem cells expressing a CXCR4 receptor.

The compositions described here may be administered by any suitable route of delivery. In embodiments, for example where the target cells are located in the brain, delivery may be by intranasal delivery.

In embodiments, the disclosure provides methods of treating glioblastoma by administering to a subject in need of therapy a composition comprising a plurality of nanovesicles, each comprising from about 500-800 copies of a mixture of miRNAs, wherein the nanovesicles of the plurality have a mean particle diameter of from about 90-150 nm. In embodiments, the mixture of miRNAs comprises a mixture of anti-miRNA-21 and miRNA-100. In embodiments, the nanovesicles comprise a recombinant membrane associated protein expressed on the external surface of the nanovesicles. In embodiments, the recombinant protein is a CXCR4 receptor. In embodiments, the methods further comprise administering temozolomide (TMZ) to the subject. In embodiments, administration of the composition comprising the plurality of nanovesicles is by intranasal delivery.

Glioblastoma (GBM) patients have an overwhelmingly poor prognosis despite multimodal therapies. Most chemotherapies fail to achieve therapeutic drug doses at tumors owing to blood-brain barrier (BBB) restrictions. More specific alternative treatments, such as use of therapeutic microRNAs (miRNAs) that can in vivo alter expression of genes associated with cancer, may provide potential opportunities to combat GBMs. MiRNA-targeted therapeutics are already in clinical translation, including miR-34 and anti-miRNA-122. By negatively regulating their target mRNAs, miRNAs can act as tumor suppressors or oncogenes (oncomiRNAs). MiRNA-21 is an oncomiRNA overexpressed in most GBMs, involved in gliomagenesis, invasion, metastasis, and regulating apoptosis and drug resistance pathways. Conversely, miRNA-100 is downregulated in GBM, resulting in tumorigenesis. Combining anti-miRNA-21 and miRNA-100 could be an advantageous therapeutic strategy to improve the effects of temozolomide (TMZ), currently the first-line clinical drug for GBM patients.

### Definitions

The following definitions are included for the purpose of understanding the present subject matter and for constructing the appended patent claims. The abbreviations used herein have their conventional meanings within the chemical and biological arts.

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

As used herein, the terms “metastasis,” “metastatic,” and “metastatic cancer” can be used interchangeably and refer to the spread of a proliferative disease or disorder, e.g., cancer, from one organ or another non-adjacent organ or body part, e.g., the brain (GGM). Cancer occurs at an originating site, e.g., brain, which site is referred to as a primary tumor, e.g., brain cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if brain cancer metastasizes to the breast, the secondary tumor at the site of the breast consists of abnormal brain cells and not abnormal breast cells.

“Patient” or “subject in need thereof” refers to a living member of the animal kingdom suffering from or who may suffer from the indicated disorder. In embodiments, the subject is a member of a species comprising individuals who may naturally suffer from the disease. In embodiments, the subject is a mammal. Non-limiting examples of mammals include rodents (e.g., mice and rats), primates (e.g., lemurs, bushbabies, monkeys, apes, and humans), rabbits, dogs (e.g., companion dogs, service dogs, or work dogs such as police dogs, military dogs, race dogs, or show dogs), horses (such as race horses and work horses), cats (e.g., domesticated cats), livestock (such as pigs, bovines, donkeys, mules, bison, goats, camels, and sheep), and deer. In embodiments, the subject is a human.

The terms “subject,” “patient,” “individual,” etc. are not intended to be limiting and can be generally interchanged. That is, an individual described as a “patient” does not necessarily have a given disease, but may be merely seeking medical advice.

The transitional term “comprising,” which is synonymous with “including” or “containing”, is inclusive or open-ended and does not exclude additional, unrecited elements or method steps. By contrast, the transitional phrase “consisting of” excludes any element, step, or ingredient not specified. The transitional phrase “consisting essentially of” limits the scope of a claim to the specified materials or steps and those that do not materially affect the basic and novel characteristics of the claimed invention.

In the descriptions herein and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

It is understood that where a parameter range is provided, all integers within that range, and tenths thereof, are also provided by the invention. For example, “0.2-5 mg” is a disclosure of 0.2 mg, 0.3 mg, 0.4 mg, 0.5 mg, 0.6 mg etc. up to and including 5.0 mg.

As used in the description herein and throughout the claims that follow, the meaning of “a,” “an,” and “the” includes plural reference unless the context clearly dictates otherwise.

As used herein, “treating” or “treatment” of a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean inhibiting the progression of the condition, disorder or disease, slowing the progression of the condition, disorder or disease temporarily, although in some instances, it involves halting the progression of the condition, disorder or disease permanently.

As used herein, the terms “treat” and “prevent” are not intended to be absolute terms. In various embodiments, treatment can refer to a 10%, 20%, 30%, 40%, 50%, 60%, 70%, 80%, 90%, or 100% reduction in the severity of an established disease, condition, or symptom of the disease or condition. In embodiments, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. In embodiments, references to decreasing, reducing, or inhibiting include a change of 10%, 20%, 30%, 40%, 50%, 60%, 70%, 80%, 90% or greater as compared to a control level and such terms can include but do not necessarily include complete elimination. In embodiments, the severity of disease is reduced by at least 10%, as compared, e.g., to the individual before administration or to a control individual not undergoing treatment. In some aspects the severity of disease is reduced by at least 25%, 50%, 75%, 80%, or 90%, or in some cases, no longer detectable using standard diagnostic techniques.

The terms “effective amount,” “effective dose,” etc. refer to the amount of an agent that is sufficient to achieve a desired effect, as described herein. In embodiments, the term “effective” when referring to an amount of cells or a therapeutic compound may refer to a quantity of the cells or the compound that is sufficient to yield an improvement or a desired therapeutic response without undue adverse side effects (such as toxicity, irritation, or allergic response) commensurate with a reasonable benefit/risk ratio when used in the manner of this disclosure. In embodiments, the term “effective” when referring to the generation of a desired cell population may refer to an amount of one or more compounds that is sufficient to result in or promote the production of members of the desired cell population, especially compared to culture conditions that lack the one or more compounds.

As used herein, an “isolated” or “purified” nucleic acid molecule, polynucleotide, polypeptide, or protein, is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (RNA or DNA) is free of the genes or sequences that flank it in its naturally-occurring state. Purified also defines a degree of sterility that is safe for administration to a human subject, e.g., lacking infectious or toxic agents.

Similarly, by “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

A “control” sample or value refers to a sample that serves as a reference, usually a known reference, for comparison to a test sample. For example, a test sample can be taken from a test subject, e.g., a subject with cancer and compared to samples from known conditions, e.g., a subject (or subjects) that does not have cancer (a negative or normal control), or a subject (or subjects) who does have cancer (positive control). A control can also represent an average value gathered from a number of tests or results. One of skill in the art will recognize that controls can be designed for assessment of any number of parameters. One of skill in the art will understand which controls are valuable in a given situation and be able to analyze data based on comparisons to control values. Controls are also valuable for determining the significance of data. For example, if values for a given parameter are variable in controls, variation in test samples will not be considered as significant.

The term, “normal amount” with respect to a compound (e.g., a protein or mRNA) refers to a normal amount of the compound in an individual who does not have cancer in a healthy or general population. The amount of a compound can be measured in a test sample and compared to the “normal control” level, utilizing techniques such as reference limits, discrimination limits, or risk defining thresholds to define cutoff points and abnormal values (e.g., for cancer or a symptom thereof). The normal control level means the level of one or more compounds or combined compounds typically found in a subject known not suffering from cancer. Such normal control levels and cutoff points may vary based on whether a compounds is used alone or in a formula combining with other compounds into an index. Alternatively, the normal control level can be a database of compounds patterns from previously tested subjects who did not develop cancer or a particular symptom thereof (e.g., in the event the cancer develops or a subject already having cancer is tested) over a clinically relevant time horizon.

The level that is determined may be the same as a control level or a cut off level or a threshold level, or may be increased or decreased relative to a control level or a cut off level or a threshold level. In some aspects, the control subject is a matched control of the same species, gender, ethnicity, age group, smoking status, body mass index (BMI), current therapeutic regimen status, medical history, or a combination thereof, but differs from the subject being diagnosed in that the control does not suffer from the disease (or a symptom thereof) in question or is not at risk for the disease.

Relative to a control level, the level that is determined may an increased level. As used herein, the term “increased” with respect to level (e.g., protein or mRNA level) refers to any % increase above a control level. In various embodiments, the increased level may be at least or about a 5% increase, at least or about a 10% increase, at least or about a 15% increase, at least or about a 20% increase, at least or about a 25% increase, at least or about a 30% increase, at least or about a 35% increase, at least or about a 40% increase, at least or about a 45% increase, at least or about a 50% increase, at least or about a 55% increase, at least or about a 60% increase, at least or about a 65% increase, at least or about a 70% increase, at least or about a 75% increase, at least or about a 80% increase, at least or about a 85% increase, at least or about a 90% increase, at least or about a 95% increase, relative to a control level.

Relative to a control level, the level that is determined may a decreased level. As used herein, the term “decreased” with respect to level (e.g., protein or mRNA level) refers to any % decrease below a control level. In various embodiments, the decreased level may be at least or about a 5% decrease, at least or about a 10% decrease, at least or about a 15% decrease, at least or about a 20% decrease, at least or about a 25% decrease, at least or about a 30% decrease, at least or about a 35% decrease, at least or about a 40% decrease, at least or about a 45% decrease, at least or about a 50% decrease, at least or about a 55% decrease, at least or about a 60% decrease, at least or about a 65% decrease, at least or about a 70% decrease, at least or about a 75% decrease, at least or about a 80% decrease, at least or about a 85% decrease, at least or about a 90% decrease, at least or about a 95% decrease, relative to a control level.

The terms “polypeptide,” “peptide” and “protein” are used interchangeably herein to refer to a polymer of amino acid residues, wherein the polymer may in embodiments be conjugated to a moiety that does not consist of amino acids. The terms also apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed or chemically synthesized as a single moiety.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. In embodiments, the percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “identical” or percent “identity,” in the context of two or more nucleic acids or polypeptide sequences, refer to two or more sequences or subsequences that are the same or have a specified percentage of amino acid residues or nucleotides that are the same (e.g., 50%, 55%, 60%, 65%, 70%, 75%, 80%, 85%, 90%, 91%, 92%, 93%, 94%, 95%, 96%, 97%, 98%, 99%, or more identity over a specified region, e.g., of an entire polypeptide sequence or an individual domain thereof), when compared and aligned for maximum correspondence over a comparison window, or designated region as measured using a sequence comparison algorithm or by manual alignment and visual inspection. In embodiments, two sequences are 100% identical. In embodiments, two sequences are 100% identical over the entire length of one of the sequences (e.g., the shorter of the two sequences where the sequences have different lengths). In embodiments, identity may refer to the complement of a test sequence. In embodiments, the identity exists over a region that is at least about 10 to about 100, about 20 to about 75, about 30 to about 50 amino acids or nucleotides in length. In embodiments, the identity exists over a region that is at least about 50 amino acids or nucleotides in length, or more preferably over a region that is 100 to 500, 100 to 200, 150 to 200, 175 to 200, 175 to 225, 175 to 250, 200 to 225, 200 to 250 or more amino acids or nucleotides in length.

For sequence comparison, typically one sequence acts as a reference sequence, to which test sequences are compared. In embodiments, when using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window” refers to a segment of any one of the number of contiguous positions (e.g., least about 10 to about 100, about 20 to about 75, about 30 to about 50, 100 to 500, 100 to 200, 150 to 200, 175 to 200, 175 to 225, 175 to 250, 200 to 225, 200 to 250) in which a sequence may be compared to a reference sequence of the same number of contiguous positions after the two sequences are optimally aligned. In embodiments, a comparison window is the entire length of one or both of two aligned sequences. In embodiments, two sequences being compared comprise different lengths, and the comparison window is the entire length of the longer or the shorter of the two sequences. In embodiments relating to two sequences of different lengths, the comparison window includes the entire length of the shorter of the two sequences. In embodiments relating to two sequences of different lengths, the comparison window includes the entire length of the longer of the two sequences.

Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Non-limiting examples of algorithms that are suitable for determining percent sequence identity and sequence similarity are the BLAST and BLAST 2.0 algorithms, which are described in Altschul et al., Nuc. Acids Res. 25:3389-3402 (1977) and Altschul et al., J Mol. Biol. 215:403-410 (1990), respectively. BLAST and BLAST 2.0 may be used, with the parameters described herein, to determine percent sequence identity for nucleic acids and proteins. Software for performing BLAST analyses is publicly available through the National Center for Biotechnology Information (NCBI), as is known in the art. An exemplary BLAST algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. In embodiments, the NCBI BLASTN or BLASTP program is used to align sequences. In embodiments, the BLASTN or BLASTP program uses the defaults used by the NCBI. In embodiments, the BLASTN program (for nucleotide sequences) uses as defaults: a word size (W) of 28; an expectation threshold (E) of 10; max matches in a query range set to 0; match/mismatch scores of 1,−2; linear gap costs; the filter for low complexity regions used; and mask for lookup table only used. In embodiments, the BLASTP program (for amino acid sequences) uses as defaults: a word size (W) of 3; an expectation threshold (E) of 10; max matches in a query range set to 0; the BLOSUM62 matrix (see Henikoff & Henikoff, Proc. Natl. Acad. Sci. USA 89:10915 (1992)); gap costs of existence: 11 and extension: 1; and conditional compositional score matrix adjustment.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

“Nucleic acid” refers to nucleotides (e.g., deoxyribonucleotides, ribonucleotides, and 2′-modified nucleotides) and polymers thereof in either single-, double- or multiple-stranded form, or complements thereof. The terms “polynucleotide,” “oligonucleotide,” “oligo” or the like refer, in the usual and customary sense, to a linear sequence of nucleotides. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness.

Nucleic acids, including e.g., nucleic acids with a phosphorothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent, or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, Oligonucleotides and Analogues: A Practical Approach, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

“Operably linked” refers to a juxtaposition wherein the components so described are in a relationship permitting them to function in their intended manner. A control sequence “operably linked” to a coding sequence is ligated in such a way that expression of the coding sequence is achieved under conditions compatible with the control sequences.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides and/or ribonucleotides, and/or analogs, derivatives or modifications thereof. Different polynucleotides may have different three-dimensional structures, and may perform various functions, known or unknown. Non-limiting examples of polynucleotides include genomic DNA, a genome, mitochondrial DNA, a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

The term “amino acid residue,” as used herein, encompasses both naturally-occurring amino acids and non-naturally-occurring amino acids. Examples of non-naturally occurring amino acids include, but are not limited to, D-amino acids (i.e. an amino acid of an opposite chirality to the naturally-occurring form), N-α-methyl amino acids, C-α-methyl amino acids, β-methyl amino acids and D- or L-β-amino acids. Other non-naturally occurring amino acids include, for example, β-alanine (β-Ala), norleucine (Nle), norvaline (Nva), homoarginine (Har), 4-aminobutyric acid (γ-Abu), 2-aminoisobutyric acid (Aib), 6-aminohexanoic acid (F-Ahx), ornithine (orn), sarcosine, α-amino isobutyric acid, 3-aminopropionic acid, 2,3-diaminopropionic acid (2,3-diaP), D- or L-phenylglycine, D-(trifluoromethyl)-phenylalanine, and D-p-fluorophenylalanine.

As used herein, “peptide bond” can be a naturally-occurring peptide bond or a non-naturally occurring (i.e. modified) peptide bond. Examples of suitable modified peptide bonds are well known in the art and include, but are not limited to, —CH₂NH—, —CH₂S—, —CH₂CH₂—, —CH═CH— (cis or trans), —COCH₂—, —CH(OH)CH₂—, —CH₂SO—, —CS—NH— and —NH—CO— (i.e. a reversed peptide bond) (see, for example, Spatola, Vega Data Vol. 1, Issue 3, (1983); Spatola, in Chemistry and Biochemistry of Amino Acids Peptides and Proteins, Weinstein, ed., Marcel Dekker, New York, p. 267 (1983); Morley, J. S., Trends Pharm. Sci. pp. 463-468 (1980); Hudson et al., Int. J. Pept. Prot. Res. 14:177-185 (1979); Spatola et al., Life Sci. 38:1243-1249 (1986); Hann, J. Chem. Soc. Perkin Trans. I 307-314 (1982); Almquist et al., J. Med. Chem. 23:1392-1398 (1980); Jennings-White et al., Tetrahedron Lett. 23:2533 (1982); Szelke et al., EP 45665 (1982); Holladay et al., Tetrahedron Lett. 24:4401-4404 (1983); and Hruby, Life Sci. 31:189-199 (1982)).

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The following examples illustrate certain specific embodiments of the invention and are not meant to limit the scope of the invention.

Embodiments herein are further illustrated by the following examples and detailed protocols. However, the examples are merely intended to illustrate embodiments and are not to be construed to limit the scope herein. The contents of all references and published patents and patent applications cited throughout this application are hereby incorporated by reference.

Further experimental details for the examples below can be found in Wang et al., ACS Nano. 2021 Nov. 1. doi: 10.1021/acsnano.1c07587, the content of which is hereby incorporated by reference.

### Example 1: CXCR4-Engineered NSC-mpEVs for Loading Therapeutic miRNAs Using Microfluidics

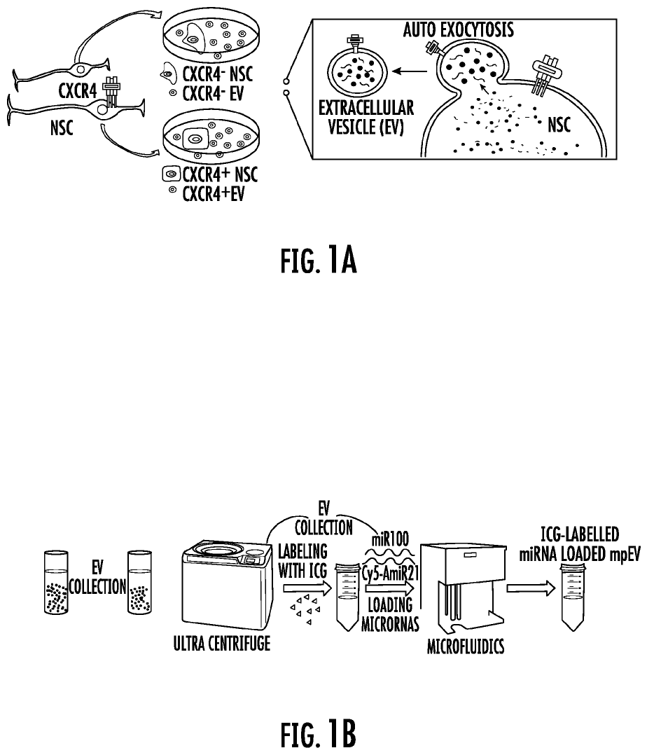

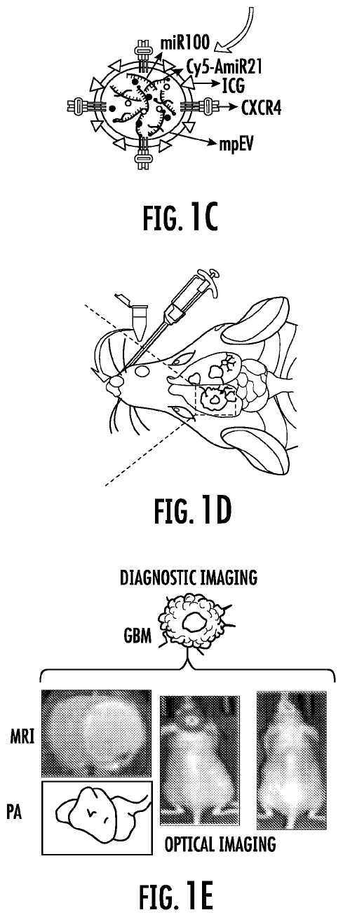

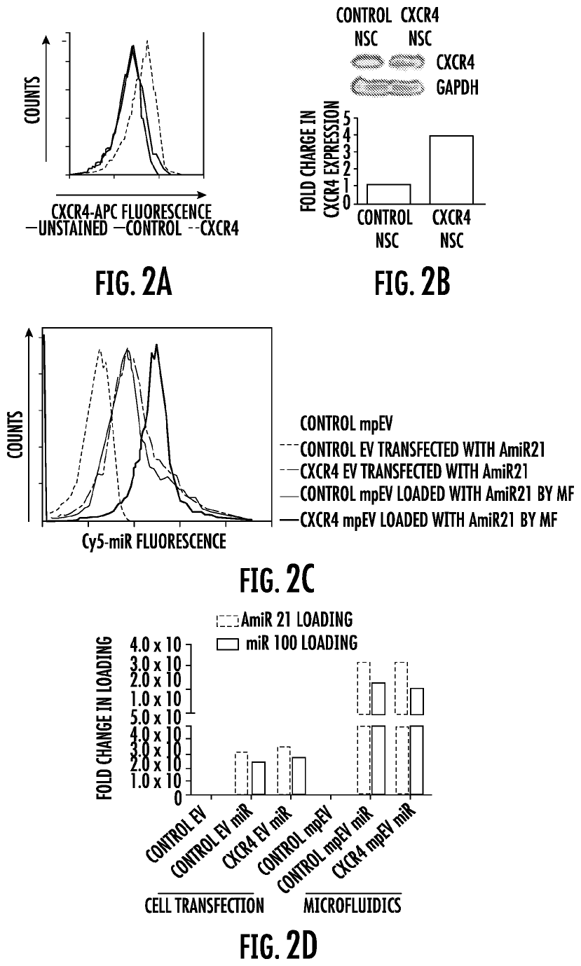

A stable and reliable miRNA delivery system was developed along with a novel administration route for therapeutic miRNA applications to GBMs. Mammalian cell-derived mpEVs were developed as delivery agents and the nose-to-brain transport pathway as an efficient delivery route to bypass the BBB. FIG. 1 is a schematic of the study workflow. In order to improve the tumor-tropic and targeting properties of our vesicles, termed “NSCEVs”, we initially engineered neural stem cells (NSCs) to overexpress the CXCR4 receptor. Glioblastomas (GBMs) inherently secrete higher amounts of the cytokine SDF-1e (CXCL12), a ligand for the CXCR4 receptor. As shown in FIG. 2A, the NSCs engineered to express CXCR4 receptors demonstrated high antibody staining signal, with a FACS fluorescence intensity shift for NSCs having successful CXCR4 surface decoration compared to control NSCs with and without CXCR4 antibody staining. Western blot quantitative analysis (FIG. 2B) revealed-′4-fold higher CXCR4 receptor expression in engineered NSCs compared to control NSCs, a finding further supported by confocal immunofluorescence imaging (not shown).

Cell-derived extracellular vesicles, or “EVs” typically will include a mixture of exosomes (30-100 nm) and microvesicles (MVs, 100-1000 nm). Cells generally produce many more MVs compared to exosomes. Considering this abundance of MVs, and with considerations for future clinical translation, the extracellular vesicles (EVs) described here were derived from MVs for use as therapeutic miRNA delivery vehicles. CXCR4-engineered NSCs were used for EV isolation and processing. We also developed a microfluidics-based reconstitution process to load miRNAs into the vesicles. As described herein, the process produces loaded vesicles of uniform size in the nanometer range and are referred to herein interchangeably as “nanovesicles” and “mpEVs”. This approach is advantageous in that it is clinically scalable and can load higher amounts of therapeutic miRNAs per vesicle while producing vesicles of a more uniform size than other methods, such as transfection based methods. This is highly relevant to future clinical applications since large quantities of therapeutic EVs are likely to be required for effective treatment. To evaluate the microfluidic processing method, we used MVs from control and CXCR4-engineered NSCs. In this context, “control” refers to cells that were not engineered to recombinantly express CXCR4. To compare the miRNA loading efficiencies, we isolated MVs from NSCs with and without miRNA transfection. The MVs isolated from NSCs without miRNA transfection (NSCs and CXCR4-NSCs) were used to load miRNAs using a microfluidic process. 10 nmols of each miRNA (Cy5-anti-miRNA-21 and miRNA-100) per 1.0×10¹⁵ MVs in 6 ml of physiological saline were subjected to processing through an LV1-microfluidic system at a pressure of 30,000 psi. The microfluidically processed MVs are referred to herein as “mpEVs”. The miRNA loaded mpEVs were enriched by ultracentrifugation and used for subsequent evaluations of EV size and surface charge using FACS, DLS, NTA and TEM analysis. To verify the presence of CXCR4 receptors on the surface of the mpEVs, we performed immunogold labelling of CXCR4-mpEVs and imaged using transmission electron microscopy (TEM). The TEM images indicated a scattered and uniform presence of gold tagged CXCR4 receptors throughout the surface of the vesicles. We also measured the extent of miRNA loaded in these vesicles using quantitative RT-PCR as compared to control EVs and EVs isolated from cells transfected with similar amounts of miRNAs.

There were no significant differences in the size and distribution of MVs isolated from control and CXCR4-engineered NSCs. The transfection-based loading of miRNAs did not affect the size and distribution of MVs. In contrast, the microfluidic-based reconstitution resulted in a homogeneous distribution of mpEVs with uniform sizes. We used Cy5-labeled miRNA to quantitatively measure the loading efficiency using FACS analysis, and found significantly higher fluorescence signal from mpEVs reconstituted using microfluidics compared to EVs isolated from transfected cells (P<0.001). The mpEVs produced by the microfluidic-based miRNA loading approach showed higher Cy5 fluorescence signal compared to control and EVs isolated from transfected cells (FIG. 2C). We further estimated the loaded miRNA levels using quantitative RT-PCR, and observed significantly higher (P<0.001) levels of miRNA loading when using the microfluidic system (FIG. 2D), that is, 10-fold higher than the loading achieved using the transfection system.

Microfluidic-mediated reconstruction of EVs to load miRNAs is one of the important features of our work. To provide a clear illustration of the advantages of this approach over transfection-mediated miRNA loading in EVs by natural biogenesis, we quantified the miRNA payload in vesicles derived from both approaches using quantitative RT-PCR to estimate their loading efficiency in terms of loaded miRNA copies per vesicle. To derive an absolute quantitation, we constructed a standard curve for anti-miRNA-21 by amplifying known amounts of anti-miRNA-21. The resultant Ct values were plotted against log (No×10⁻¹) to draw a correlation between Ct number and anti-miRNA-21 copy number using curve fitting (FIG. 9). We used the corresponding Ct values from different conditions to derive a total number of miRNA copies loaded in these vesicles. Likewise, we determined the concentration of EVs/mpEVs using NTA, and used this to determine the number of miRNA copies loaded per EVs/mpEVs.

The standard plot for anti-miRNA-21 generated fitting line was represented by:

$$Log ( No \times 0.1 ) = 15.3518 - 0.3938 Ct$$

Where “No” is the initial miRNA copy number and Ct is the cycle threshold value obtained from the PCR reaction.

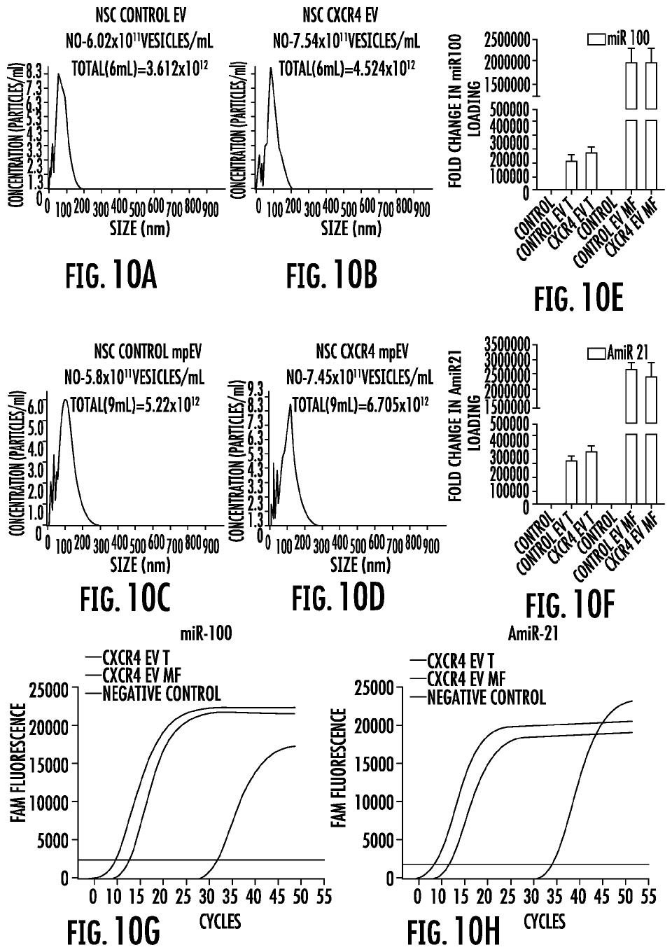

We used equivalent amounts of EVs (control or CXCR4) for microfluidic reconstruction and miRNA loading as that of EVs (control or CXCR4) harvested from miRNA (20 nmols) transfected NSCs. The microfluidic mediated reconstruction of EVs resulted in an increase in number of mpEVs in both cases (control EVs increased from 3.612×10¹² vesicles to 5.22×10¹² mpEVs, and CXCR4-EVs increased from 4.524×10¹² EVs to 6.705×10¹² mpEVs). The increase in vesicle number upon microfluidic processing matched the finding of relatively uniform and smaller sizes of resulting mpEVs. The total input of miRNA in all cases was 20 nmols, which corresponded to 1.2×10¹⁶ copies of anti-miRNA-21. The amplification curves for anti-miRNA-21 extracted from EVs/mpEVs are shown in FIG. 10G-H. As evident in both CXCR4 and control EVs, the microfluidic processing increased miRNA loading efficiency by nearly 8-fold to 10-fold compared to EVs isolated from transfected cells. This observation was consistent for miRNA-100 quantification for EVs and mpEVs of control and CXCR4-NSCs (FIG. 10E and FIG. 10F). The observed increase in loading efficiency was calculated using an anti-miRNA-21 standard curve and is tabulated in Table 1 below.

TABLE 1
Quantitative estimation of loaded anti-miR-21 in EVs isolated from transfected cells
(EV T) and mpEVs processed by microfluidics

| | | | | | | Absolute | Loading | | miR/ |
|---|---|---|---|---|---|---|---|---|---|
| | Input | | | | | miR | effici- | | vesicle |
| | microRNA | RT-PCR | | Copy | Dilution | (copy | ency | No of | (copy |
| Sample | (nmol) | (Ct) | Log(Nox).1) | no. | factor | no.) | (%) | vesicles | no.) |
| Control | 20 | 10.9450440 | 11.0396416 | 1.096 × | 500 | 5.48 × | 4.566 | 3.612 × | 151 |
| EV T | | | | 10\hat{ }12 | | 10\hat{ }14 | | 10\hat{ }12 | |
| CXCR4 | 20 | 10.6341768 | 11.1622611 | 1.452 × | 500 | 7.264 × | 6.054 | 4.524 × | 160 |
| EV T | | | | 10\hat{ }12 | | 10\hat{ }14 | | 10\hat{ }12 | |
| Control | 20 | 7.73227260 | 12.3050310 | 2.018 × | 200 | 4.036 × | 33.64 | 5.22 × | 773 |
| mpEV | | | | 10\hat{ }13 | | 10\hat{ }15 | | 10\hat{ }12 | |
| CXCR4 | 20 | 7.73154679 | 12.3053168 | 2.019 × | 200 | 4.039 × | 33.66 | 6.705 × | 602 |
| mpEV | | | | 10\hat{ }13 | | 10\hat{ }15 | | 10\hat{ }12 | |

While transfection of miRNA in source cells can load nearly 100 copies of miRNA in each vesicle during natural biogenesis processing, the use of microfluidic-mediated reconstruction of EVs increased the loaded miRNA copies to 600-775 per vesicle. Apart from achieving higher amounts of miRNA copies in each vesicle, the microfluidic approach also achieves an encapsulation efficiency of 33% as opposed to a mere 4-6% when using a transfection-mediated approach. Thus, the strategy outlined in this study improved the overall extent of miRNA loading by nearly 8-fold to 10-fold compared to transfection.

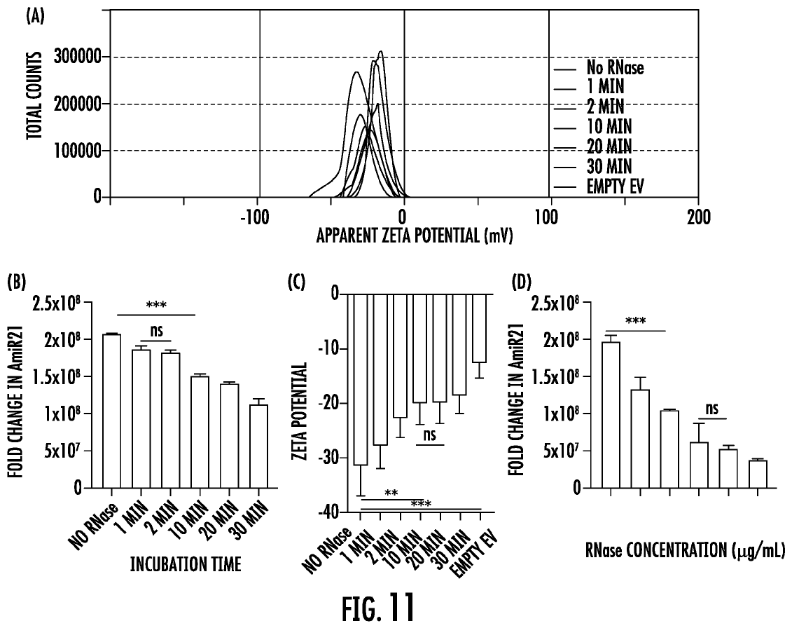

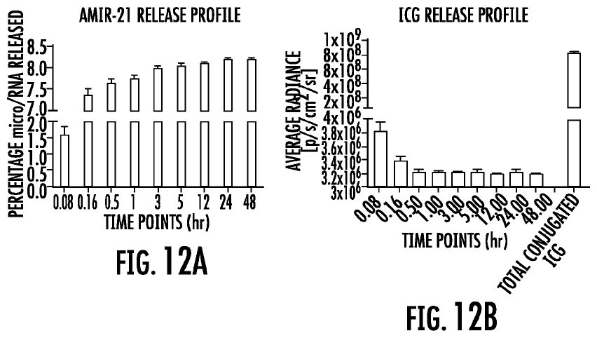

With established values of miRNA loading efficiency for mpEVs, we further evaluated if these loaded miRNAs were surface bound or loaded within the vesicles based on their susceptibility to degradation by performing an RNase protection assay (FIG. 11). The experimental methods, and the results and discussion of the RNase protection assay are provided below. Further, in tandem with miRNA stability, we also performed time dependent release profiles of miRNAs from these vesicles under physiological conditions, as shown in FIG. 12 and discussed below.

### Characterization of Labeled NS-mpEVs

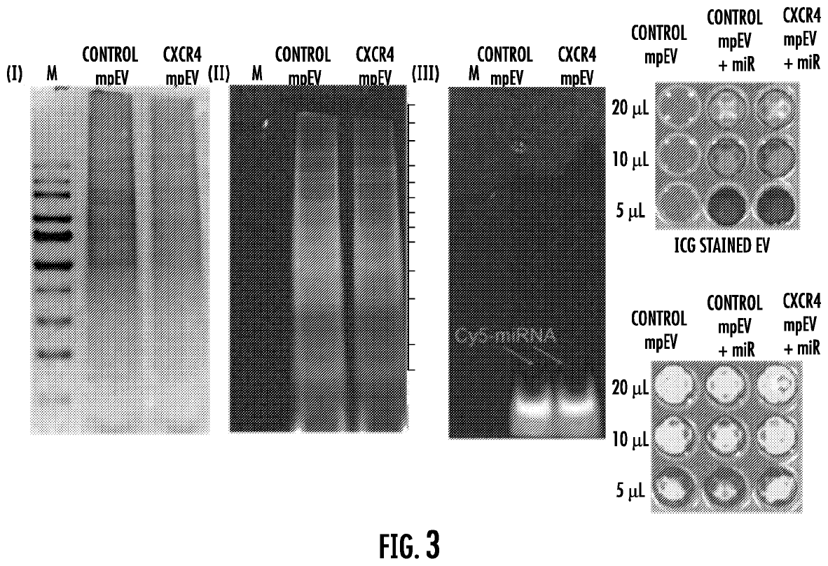

To facilitate visual tracking using optical and photoacoustic imaging, and to ascertain delivery efficiency in cells using microscopic analysis, or after intranasal treatments in vivo in mice, we used ICG as a fluorophore. ICG emits light in the NIR window and possesses photoacoustic imaging properties. We labeled EV membranes using ICG by an amine conjugation technique. After removing unconjugated ICG dye by ultracentrifugation, we used ICG labeled EVs for miRNA loading using microfluidics. In order to test the labeling efficiency of ICG to different proteins of the EV membrane, we resolved the EV proteins in SDS gel electrophoresis and imaged using optical fluorescence imaging. FIG. 3 shows the (i) Coomassie blue stained gel for the presence of proteins of different molecular weights in mpEVs; (ii) the image of the same gel acquired under an ICG filter (Ex740 nm/Em830 nm) for the labeled mpEVs, and (iii) Cy5 filter (Ex630 nm/Em650 nm) for the loaded Cy5-miRNA-21. The in vitro NIR and ICG labeling of mpEVs shown in panel (iv) reveals an equal ICG labeling and loading efficiency of miRNAs in both control and CXCR4-NSC-mpEVs, as well as the concentration dependent signal intensity of ICG and Cy5 dye from both types of NSC-mpEVs. The uniformity of ICG labeling and Cy5-miRNAs loading levels in control and CXCR4-mpEVs was supported by FACS analysis. In addition, NTA analysis revealed there was no significant difference in the size of NSC-mpEVs before and after loading miRNAs using the microfluidic device and engineered to express CXCR4. The zeta potential of surface membrane of NSC-mpEVs was also analyzed using DLS. As expected, the mpEVs loaded with miRNAs showed slight increase in negative zeta potential compared to those without miRNAs. Overall, these results support the efficiency of microfluidic processing, ICG labeling, and mpEVs of uniform characteristics in both type of NSC-mpEVs, making them appropriate for tracking the delivery of mpEVs in vitro using microscopy, by optical and photoacoustic imaging in vivo, and for scalable comparison and assessment between different treatment groups in mice.

Labeling of mpEVs Using ICG-NIR Dye to or In Vitro and In Vivo Tracking of miRNA-Loaded mpEVs

To facilitate visual tracking using optical and photoacoustic imaging, and to ascertain delivery efficiency in cells using microscopic analysis, or after intranasal treatments in mice, we used the ICG fluorophore for labeling mpEVs (FIG. 3). After the characterization of mpEVs, we evaluated the microfluidics-processed miRNA-loaded mpEVs for intracellular delivery and function in glioma cells in vitro. We used NSC-mpEVs labeled with ICG dye and co-loaded with Cy5-anti-miRNA-21 and miRNA-100 in 3D spheroids of U87MG cells. The 3D images acquired using the laser confocal microscopic imaging system (TCS SP8, Leica) showed significantly higher levels of transfection using the NSC-mpEVs engineered to overexpress CXCR4 receptors compared to the basal level expression of control NSC-mpEVs.

Next, to evaluate the transfection and functional therapeutic effects of EV-delivered miRNAs in combination with TMZ, we used GL26 mouse GBM cells engineered to stably express the green fluorescent protein (GFP) and U87MG human glioma cells in a 2D culture model. We incubated the dual-fluorophore-labeled (Cy5-miRNA and ICG membrane) NSC-mpEVs with GL26 glioma cells for 48 h, followed by confocal microscopy imaging. In all five groups treated with dual-fluorophore-labeled mpEVs, there was an overlap of ICG and Cy5-miRNA fluorescence signal, in addition to GFP expressed by the cells, demonstrating the efficient delivery of the miRNA-loaded mpEVs to glioma cells. We found fluorescence signal intensity of ICG from CXCR4-NSC-mpEVs to be slightly higher than that from control NSC-mpEVs; and similar patterns were observed in Cy5 fluorescence intensity changes. This difference in fluorescence intensity was attributable to the increased delivery of mpEVs to GL26 cells mediated by the CXCR4 specific binding to their secreted CXCL12, which was upregulated upon treating with TMZ. Notably, among all the different treatment groups, GL26 glioma cells upon treating with TMZ plus miRNA-loaded CXCR4-NSC-mpEVs showed the highest ICG and Cy5 fluorescence signal intensity. Single cell analysis of the intracellular delivery of miRNA-loaded mpEVs in a single GL26 glioma cell in which the Z-axis direction was viewed to reveal NSC mpEVs-miRNA loading clearly indicated that EV-delivered miRNAs are predominantly located in the cytoplasm rather than the nucleus.

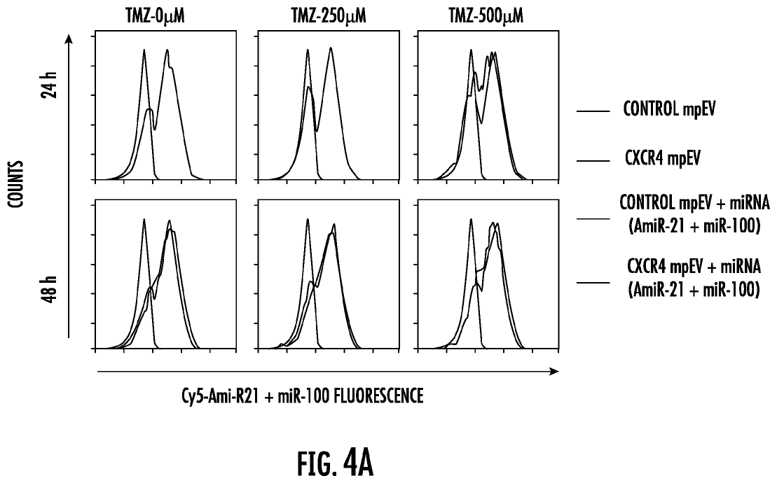

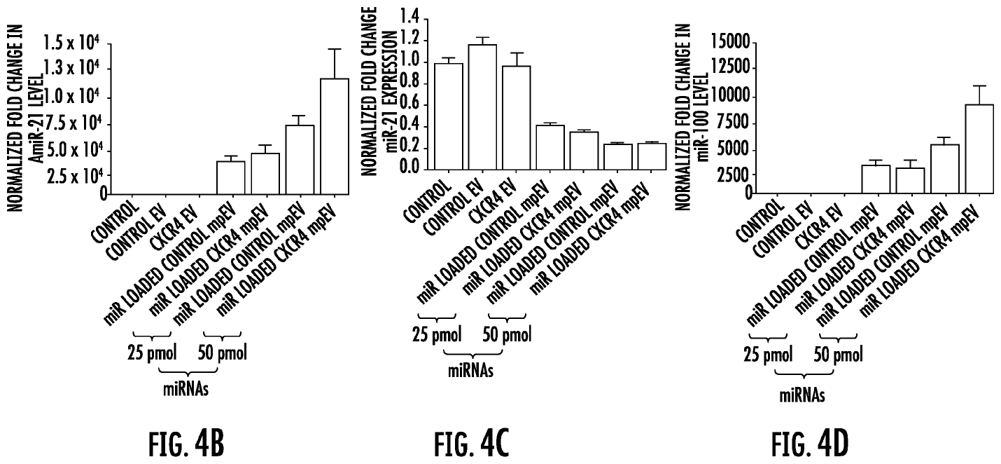

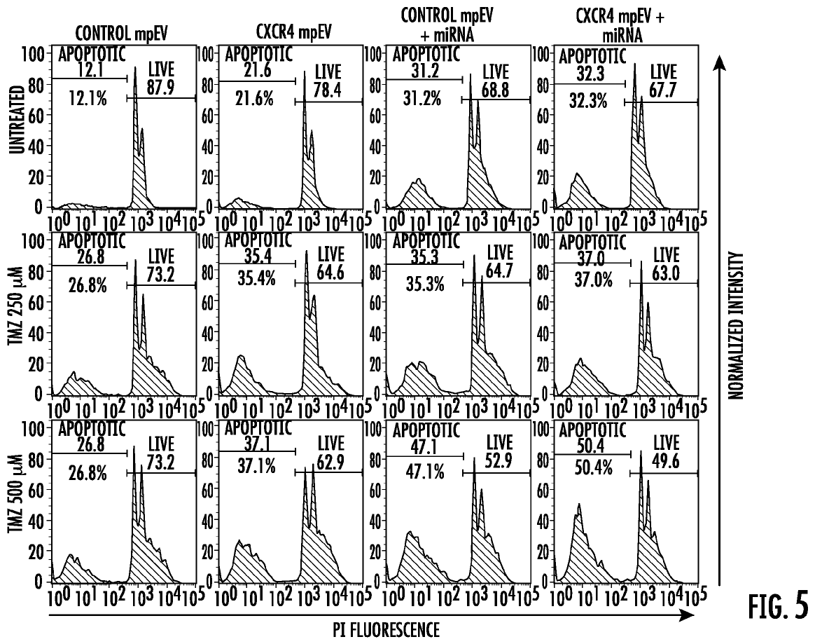

To further assess the intracellular delivery of miRNA-loaded mpEVs in glioma cells in vitro upon TMZ treatment, we performed FACS analysis at different time points (24 h and 48 h) after treatment. We divided U87MG glioma cell samples into six groups based on the TMZ administration doses (0 μM, 250 μM, and 500 μM) and time points (24 h and 48 h) (FIG. 4A). We found significant levels of delivery at both time points independent of treatment conditions. Next, we evaluated the quantity of miRNAs that were successfully delivered into cancer cells by the mpEVs in vitro. In addition to the therapeutic anti-miRNA-21 and miRNA-100 used, we also quantified the levels of endogenous miRNA-21 expression in EV-treated cells to demonstrate the biological suppression effect of the anti-miRNA-21. We quantified the extent of miRNAs using qRT-PCR at 48 h after treating U87MG cells with miRNA-mpEVs. We developed three testing groups: (1) mpEVs without loaded miRNAs, (2) mpEVs loaded with 25 pmol of miRNAs, and (3) mpEVs loaded with 50 pmol of miRNAs. We used control NSC-mpEVs and CXCR4-NSC-mp EVs. The miRNA quantification revealed an ˜3.0-3.5×10⁶-fold (25 pmol) and ˜6.5×10⁶ to 1.1×10⁷-fold (50 pmol) increase of anti-miRNA-21 levels in cells treated with control mpEVs and CXCR4-NSC-mpEVs, respectively, compared to control groups (FIG. 4B). Correspondingly, the level of endogenous miRNA-21 decreased by a ˜60% (25 pmol) and 80% (50 pmol) in groups treated using low- and high-dose miRNAs, respectively, compared to non-miRNA control mpEVs groups (FIG. 4C). As for the miRNA-100 levels delivered into U87MG tumor cells, we found an ˜2.5×10³-fold (25 pmol) and ˜5.0×10³ to 1.0×10⁴-fold (50 pmol) increase in miRNA-100 levels in groups receiving low- and high-dose miRNAs, respectively (FIG. 4D). We further compared the difference in quantities of intracellular miRNAs delivered using control NSC-mpEVs and CXCR4-NSC-mpEVs. In the high miRNAs dose group (50 pmol), the intracellular quantities of anti-miRNA-21 and miRNA-100 delivered using CXCR4-NSC-mpEVs were significantly higher (P<0.001) than those transported by control NSC-mpEVs; whereas, the difference was not significant in the low miRNA dose group (25 pmol).

### Example 2: Chemosensitizing Effects of miRNA-Loaded mpEVs on GBM Cells In Vitro

To further evaluate the therapeutic potential of miRNA-loaded mpEVs, we used U87MG cells to compare the combined therapeutic effects of TMZ and miRNAs using cells treated with different quantities of miRNAs (25 pmol and 50 pmol) loaded into NSC-mpEVs. Upon combination treatment using low-dose TMZ (250 μM), the U87MG cells showed morphologic contraction, dot-like reshaping, and detachment from the culture plate. In addition, the propidium iodide (PI) fluorescence intensity of apoptotic cells increased, corresponding to the increasing amounts of miRNAs (from 25 pmol to 50 pmol), along with the increase in ICG fluorescence intensity of miRNA-loaded mpEVs, indicating the gradually enhanced therapeutic effects of the combination treatment. Specifically, in both the low (25 pmol) and the high (50 pmol) miRNA dose groups, miRNA-loaded mpEVs from CXCR4-NSC-mpEVs demonstrated a more overt cell killing effect compared to their control counterparts. However, in the absence of anti-miRNA-21 and miRNA-100, the TMZ-mediated increase in apoptosis was not significant (P>0.05).

To quantify the chemosensitizing effects of the miRNA-loaded mpEVs, we measured apoptosis rates of U87MG cancer cells using PI staining-based FACS analysis. When U87MG cells were treated without TMZ, the percentage of apoptosis induced by CXCR4-miRNA-NSC-mpEVs was higher (32.3%) than that in cancer cells treated with CXCR4-NSC-mpEVs without loaded miRNAs (21.6%) (FIG. 5). Similarly, the U87MG cells treated with control NSC-mpEVs, with and without miRNAs, had 31.2% and 12.1% apoptotic populations, respectively, confirming the therapeutic effects of the delivered anti-miRNA-21 and miRNA-100. When co-treated with TMZ at 250 μM, the percentage of apoptosis in cancer cells treated with NSC-mpEVs without and with loaded miRNAs showed a gradual increasing trend in apoptosis (26.8 to 35.3%, respectively), which is slightly higher than their counterparts in groups without TMZ. When the TMZ dose was increased to 500 μM, the cells co-treated with TMZ and miRNA-NSC-mpEVs demonstrated 47.1 and 50.4% of apoptotic populations, respectively, by miRNA-loaded control-NSC-mpEVs and CXCR4-NSC-mpEVs, which were the highest in all treatment groups. In groups treated with control-NSC-mpEVs without loaded miRNAs, however, there were no obvious increases in apoptosis rates of U87MG cells as the TMZ dose increased from 250 μM to 500 μM. The tendency of increasing apoptosis rates for cancer cells when co-treated with TMZ and miRNA-NSC-mpEVs from both control and CXCR4-NSC-mpEVs supported the synergistic effect of anti-miRNA-21 and miRNA-100 assisted enhancement of therapeutic effects of TMZ in U87MG GBM cells.

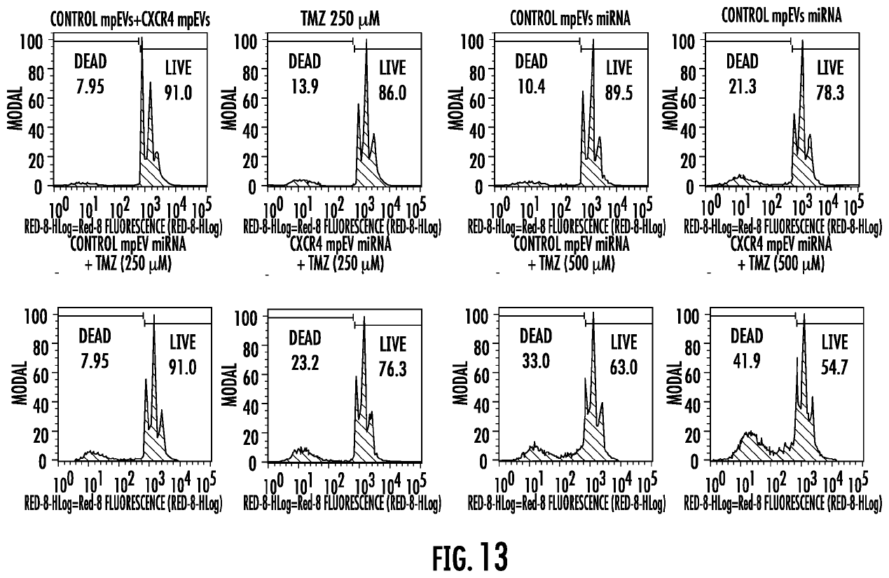

We evaluated the therapeutic potential of miRNA-loaded mpEVs (50 pmol) in GL-26 cells in combination with TMZ and quantified the chemosensitizing effects of the miRNA-loaded mpEVs using PI staining-based FACS analysis (FIG. 13). When GL-26 cells were treated with CXCR4-miRNA-mpEVs alone, the percentage of induced apoptosis was ˜10.9% higher, compared to cells treated with miRNA-loaded control-mpEVs. It is evident from this difference that increased delivery of miRNAs by CXCR4-mpEVs led to a surge in apoptotic cells which also confirmed the therapeutic effects of the delivered anti-miRNA-21 and miRNA-100 combination against GL26 cells. On the other hand, TMZ alone at 250 μM resulted in 13.9% apoptotic cells, whereas when used in combination with miRNA loaded NSC-mpEVs the extent of apoptosis increased to 23.2%. Although the chemosensitizing effects of miRNAs towards TMZ therapy were not so obvious at 250 μM TMZ, when the TMZ dose was increased to 500 μM, the cells co-treated with TMZ and miRNA-mpEVs demonstrated significant increases in apoptosis (control mpEV-miRNA+TMZ: 33% and CXCR4-mpEV-miRNA+TMZ: 41.9%), which were the highest in all treatment groups. The trend in increase of apoptotic cells when co-treated with TMZ and miRNA-mpEVs from both control and CXCR4-mpEVs supported the synergistic effects of anti-miRNA-21 and miRNA-100 assisted enhancement of therapeutic effects of TMZ in GL-26 GBM cells.

### Example 3: Therapeutic Efficacy of Intranasally Delivered miRNA-Loaded NSC-mpEVs Combined with TMZ in U87MG Orthotropic GBM Model

Next, we assessed the delivery and therapeutic efficacy of anti-miRNA-21 and miRNA-100 co-loaded NSC-mpEVs in vivo in the presence of TMZ. We used an intranasal delivery approach of loaded NSC-mpEVs in mice bearing orthotopic xenografts of U87MG GBM cells stably expressing a firefly luciferase-green fluorescence protein (FLuc-GFP) fusion reporter protein. This facilitated the monitoring of tumor growth and therapeutic response in vivo by using optical imaging. The fluorescence of the ICG dye tagged to the mpEVs also enabled real-time imaging of therapeutic miRNAs delivery in vivo.

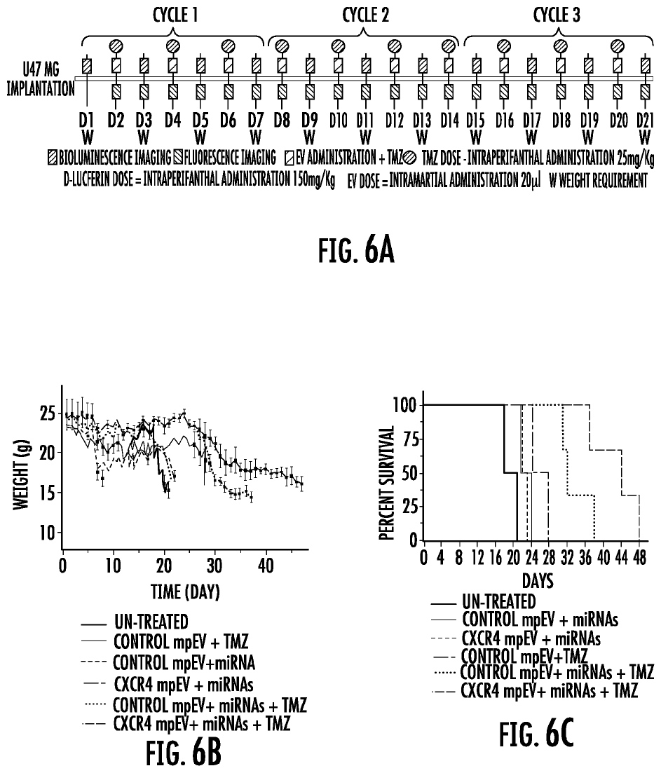

We evaluated six treatment groups: (1) Untreated (n=5); (2) Control NSC-mpEVs plus TMZ (n=5); (3) Control miRNA-NSC-mpEVs (n=5); (4) miRNA-loaded CXCR4-NSC-mpEVs (n=5); (5) Control miRNA-NSC-mpEVs plus TMZ (n=5); and (6) miRNA-loaded CXCR4-NSC-mpEVs plus TMZ (n=5). The mice in TMZ treatment groups were given the drug at a fixed dose of 25 mg/kg every other day, starting on the day FLuc bioluminescence signal from a developing U87MG-GBM tumor was observed (˜7 days after implantation). The miRNA-NSC-mpEVs were delivered intranasally in mice on the same day as TMZ administration. The schematic FIG. 6A shows the complete treatment schedule. In all six groups, the tumor associated bioluminescence signals continued to increase at different rates in the first seven days after treatment initiation.

Specifically, in the untreated group (group 1) and the other three groups without combination treatments (groups 2-4), the bioluminescence signal increased rapidly, while the signals in groups co-treated with miRNA-NSC-mpEVs plus TMZ (groups 5-6) showed relatively less intense signals. Among these, the mice receiving miRNA-loaded CXCR4-NSC-mpEVs plus TMZ had the lowest signal.

On the other hand, acquisition of ICG fluorescence images every day after the start of intranasal NSCEV delivery showed consistent patterns of EV trafficking in vivo. The miRNA-NSC-mpEVs initially accumulated in the nasal cavity owing to their mucoadhesive nature. They were then gradually absorbed through the nasal mucosa presumably to enter perivascular spaces and via branches of the olfactory and trigeminal nerves and finally migrated into the intracranial cavity and arrived at the tumor site. After 24 h of miRNA-mpEVs administration, fluorescence imaging clearly indicated the progressive accumulation of these nanocarriers in the forebrains of mice, and this could last for the following one to two days. As the treatment continued, the bioluminescence signal in groups co-treated with miRNA-mpEVs plus TMZ began to decline slightly over the treatment duration, starting at ˜8 days after treatment, most pronounced in the group treated with miRNA-loaded CXCR4-NSC-mpEVs plus TMZ. In contrast, the bioluminescence signal in the other four groups showed little visible changes or only slight signal drops. The mice co-treated with miRNA-loaded CXCR4-NSC-mpEVs plus TMZ demonstrated more pronounced declines in bioluminescence signals than those of the other five treatment groups, which also corresponded to more intense ICG fluorescence signals at the tumor site, confirming successful EV delivery and targeting. Moreover, the untreated mice experienced the shortest survival time. In contrast, the mice co-treated with miRNA-NSC-mpEVs plus TMZ demonstrated inhibition of tumor growth and also survived beyond 45 days, especially observed in mice co-treated with miRNA-loaded CXCR4-NSC-mpEVs plus TMZ (FIG. 6B and FIG. 6C).

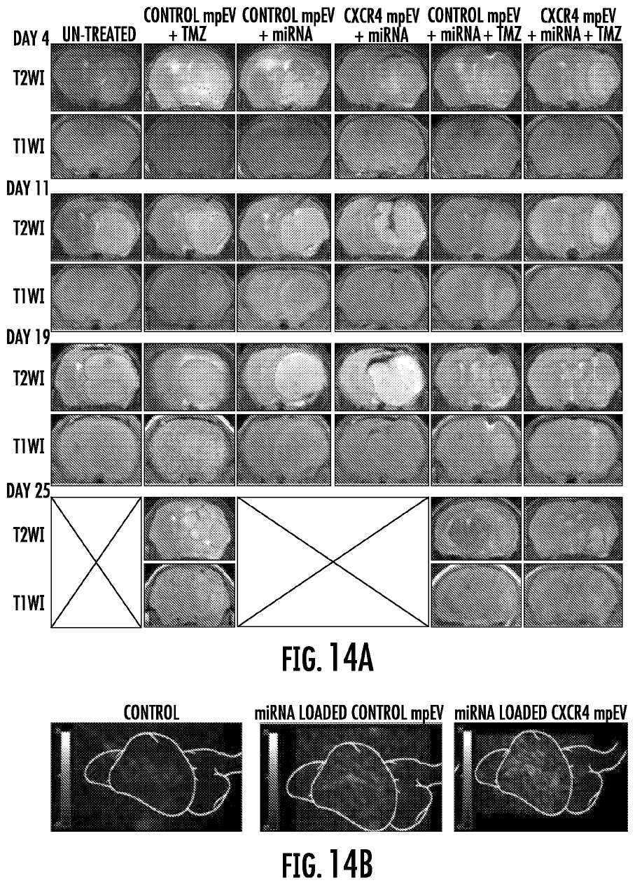

At the experimental end points, we sacrificed the mice from each treatment group and immediately harvested their organs (brain, lung, heart, liver, spleen, and kidneys), and performed ex vivo bioluminescence and fluorescence imaging. The ICG fluorescence signal showed the accumulation of miRNA-NSC-mpEVs in the brains, as well as in a few other organs. The intracranial ICG fluorescence signal of NSC-mpEVs was predominantly distributed in the rostral and forebrain regions of the brains, further supporting the notion that intranasally administered NSC-mpEVs had migrated along the pathways described earlier. We also performed periodical magnetic resonance imaging (MRI) to measure the tumor volume at four time points during treatment. The results shown in FIG. 14 clearly support the treatment response of a combination of miRNA-loaded CXCR4-NSC-mpEVs plus TMZ (group 6). Tumor progression led to body weight loss for mice prior to their ultimate demise. The weight loss was significantly delayed in the groups with TMZ plus miRNA-NSC-mpEVs, implying suppression of tumor growth (FIG. 6C).

### Example 4: Therapeutic Efficacy of Intranasally Delivered miRNA-Loaded NSC-mpEVs Combined with TMZ in GL26 GBM in an Immunocompetent C57/6J Syngeneic Mice Model

We also validated the outcome of the study in the GL26 orthotopic brain tumor model in immunocompetent C57/BL6J mice and evaluated therapeutic outcomes of treatment groups as adopted in our earlier experiments. We delivered the miRNA-loaded mpEVs intranasally in mice. The miRNA delivery and TMZ administration were followed as indicated in FIG. 6A. The delivery of miRNA-loaded mpEVs, tumor growth, and therapeutic response were tracked using ICG fluorescence imaging, tumor bioluminescence imaging, and MRI head scans at specific time points of the study. We used three animals for each treatment group and the mice survival for each treatment group was represented as a Kaplan-Meier curve.

Comparison of tumor bioluminescence signal in different treatment groups imaged at Days 4 and 20 indicated a trend in tumor progression for respective treatment groups. The tumor bioluminescence in mice administered with miRNA-loaded CXCR4-mpEVs and TMZ declined by 8-fold, whereas in the case of the control group or miRNA alone delivered group (i.e., miRNA-loaded control mpEVs and miRNA loaded CXCR4-mpEVs) we observed no significant reduction in tumor bioluminescence. We also tracked the nose-to-brain transport of ICG labeled mpEVs using ICG fluorescence imaging (745/830 nm) at corresponding time points to correlate the treatment outcomes with the extents of mpEVs delivery. In comparison to nude mice, the C57BL/6J mice exhibited a significant attenuation in ICG signal owing to depilation induced skin pigmentation as well as hair growth in the head region that limited our ability to track migration from nose to brain when using in vivo imaging. However, upon removal of head hair, the accumulation of ICG labeled mpEVs was evident in the treatment groups which validated the strategy used in this work. In vivo fluorescence imaging revealed that CXCR4-NSC-mpEVs exhibited significantly higher levels of intracranial accumulation when compared to control mpEVs. The increased accumulation of miRNA-loaded CXCR4-NSC-mpEVs in combination with TMZ treatment led to a prominent decline in tumor bioluminescence, indicating the reduction in viable tumor volume. Likewise, MRI head scans of mice also correlated well with the viable tumor volume deduced from bioluminescence imaging in the respective treatment groups. Nose-to-brain delivery of miRNA-loaded CXCR4-NSC-mpEVs in combination with TMZ led to reduction in brain tumor volume. Ex vivo bioluminescence imaging of tissues harvested from each treatment group indicated end point biodistributions of mpEVs in each treatment groups. The CXCR4-mediated targeted delivery of mpEVs was evident by higher ICG fluorescence signal in the brain. Similarly, ex vivo bioluminescence imaging of brain revealed smaller viable tumor masses in mice treated with miRNA-loaded CXCR4-NSC-mpEVs in combination with TMZ treatment, which, again, validated the therapeutic outcome of increased miRNA delivery achieved in this treatment group.

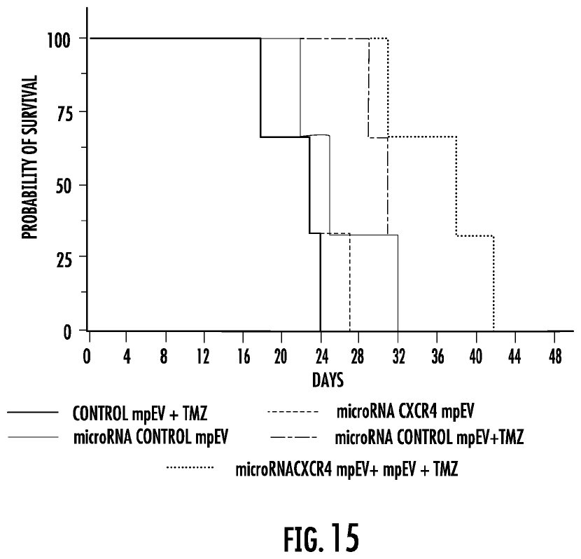

As a result of the tumor regression achieved by increased delivery of miRNAs using CXCR4-NSC-mpEVs, the therapeutic outcome was also apparent in terms of animal survival as shown by the survival curves in FIG. 15. Mice in the control group had a mean survival of 22 days, which was prolonged to a mean survival of 38 days in mice administered with CXCR4-NSC-mpEV-miRNAs in combination with TMZ. The findings of GL26 orthotopic brain tumor model established in C57/BL6J mice for different treatment groups also correlated well with the U87MG orthotopic GBM model in nude mice in terms of delivery and therapeutic outcomes.

### Example 5: Validation of Intracranial Delivery and Distribution of miRNA-NSC-m EVs Via the Nose-to-Brain Pathway

In addition to bioluminescence and fluorescence imaging, we also used photoacoustic imaging to assess the delivery of ICG-labeled miRNA-NSC-mpEVs into the brains of mice. We acquired photoacoustic images in a dorsal-to-ventral coronal direction, and demonstrated these in a sagittal view. The intensity of ICG fluorescence signal was greatest in the brains receiving the miRNA-loaded CXCR4-NSC-mpEVs plus TMZ treatment, followed by those receiving miRNA-loaded control mpEVs plus TMZ, while those treated with control NSC-mpEVs and TMZ showed a weak signal. In addition, the signal intensities in the forebrain regions were more pronounced than in dorsal brain regions, corresponding to tumor locations. Photoacoustic images indicated that miRNA-NSC-mpEVs could be successfully delivered into the brains of tumor-bearing mice after intranasal administration, and they accumulated at tumor sites.

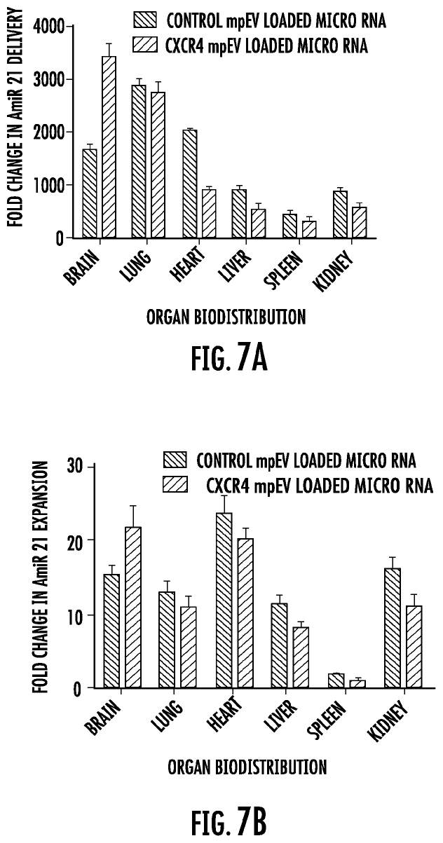

We quantified the levels of miRNAs delivered to the brains and other major organs of mice using qRT-PCR for both anti-miRNA-21 and miRNA-100. This revealed the presence of ˜3.4×10³±2.1×10²-fold of anti-miRNA-21 (endogenously absent) and ˜22±3-fold increase in miRNA-100 in the brains of mice treated with miRNA-loaded CXCR4-NSC-mpEVs. However, in mice treated with miRNA-loaded control-NSC-mpEVs, we observed only ˜1.7×10³±1.1×10²-fold of anti-miRNA-21 levels, and ˜16±2-fold increase in miRNA-100 levels, indicating that CXCR4-mediated targeting strongly improved NSC-mpEVs delivery. In addition to the majority of administered miRNA-loaded NSC-mpEVs targeted to brain GBMs, some were also detected in other organs. Quantitative analysis of anti-miRNA-21 showed their presence in lungs (79.4%), heart (29.4%), liver (17.6%), spleen (8.8%), and kidneys (20.1%) with respect to quantities in brain (100%). For miRNA-100, their presence was in lungs (50.2%), heart (90.9%), liver (38.6%), spleen (4.5%), and kidneys (51.3%) with respect to quantities in brain (100%) (FIG. 7A). In groups i-iv, the cancer cell populations densely stained with heterogeneously shaped nuclei and high incidences of dividing nuclear morphology. On the other hand, in cohorts co-treated with miRNAs-loaded NSC-mpEVs plus TMZ the tumor growth was significantly reduced. Although the NSC-mpEVs accumulated in lungs, heart, liver, spleen, and kidneys after intranasal administration, H&E histological sections did not show any hallmarks of damage in these major organs.

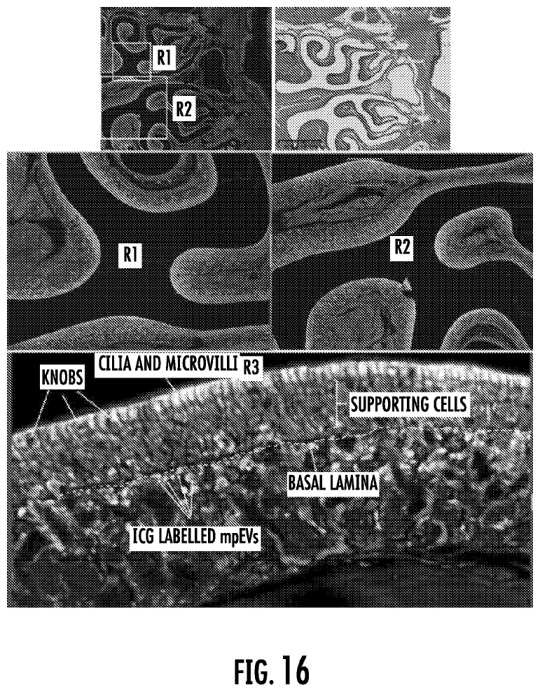

To further delineate the intranasal delivery and uptake of ICG-labeled NSC-mpEVs by nasal mucosal cells, and their subsequent trafficking into the forebrains of mice, we performed multi-fluorescence laser confocal microscopy of axial histologic slices across the nasal cavity and forebrain for the same mouse. Upon intranasal administration of mpEVs, they layer onto the olfactory mucosa (olfactory neuroepithelium) and the respiratory epithelium of the nasal cavity. Two different mechanisms come into play in the transport of mpEVs across the mucosal membranes into the olfactory bulb or main trigeminal nerves of the brain, defined as paracellullar or transcellular mechanisms. The paracellular route indicates the transport of mpEVs between the outer membranes of cells and the cilia of the mucosal membrane. The integrity of the nasal epithelium, along with the tight junctions, desmosomes, adherent junctions, and spaces between the epithelial cells allow the entry of mpEVs by paracellular transport. On the other hand, the transcellular route refers to carrier-mediated transport or endocytosis, occurring predominantly by intervening olfactory nerve receptors present in the cilia that extend to the other side of the cribriform plate into the olfactory bulb of the brain. Such intracellular transport involves receptor-mediated endocytosis or passive diffusion through the membrane of the sustenacular cells and the cells comprising the Bowman's glands along the mucosal membrane linings. Upon crossing the mucosal membrane, mpEVs arrive at the lamina propria, and ride on olfactory or trigeminal neurons to gain access to the brain. A clear evidence of such events of paracellular and transcellular transport was captured in FIG. 16 to highlight transcellular and paracellular transport of mpEVs across mucosal membranes. The section shows the pseudostratified olfactory epithelium comprising of supporting cells, olfactory sensory neurons and basal cells. The characteristic feature of supporting cells with columnar cell bodies and microvilli at the apical region of the epithelium that account for paracellular transport are clearly visible. Nanoscale mpEVs enter the perineural spaces of this cell layer through the leaky, loosely adherent epithelium surrounding the olfactory nerves or by paracellular diffusion to the underlying supporting cells. On the other hand, the dendritic knobs junctions through which olfactory neurons protrude into the nasal cavity provide a transcellular route of mpEVs and are also dispersed along the lining. The presence of intact olfactory mucosa and evidence of ICG-labeled mpEVs distinctly accumulated across this stratified structure confirm the role of paracellular and intracellular transport of mpEVs.

### Time-Lapse Quantitative Biodistribution of the EVs in Intracranial Tumor and Brain Tissue

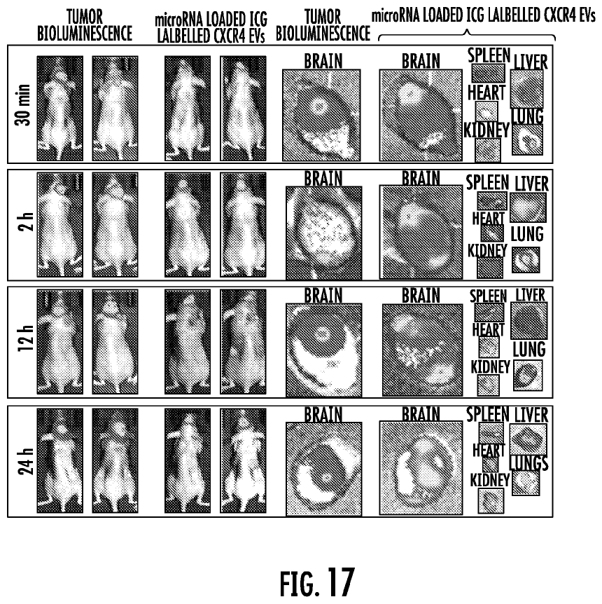

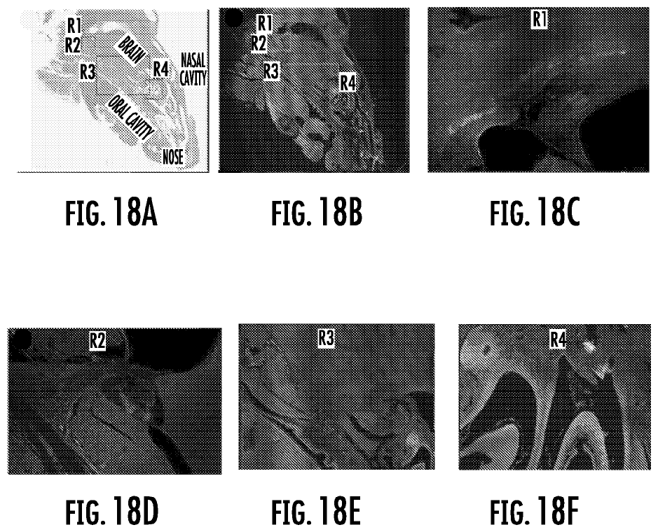

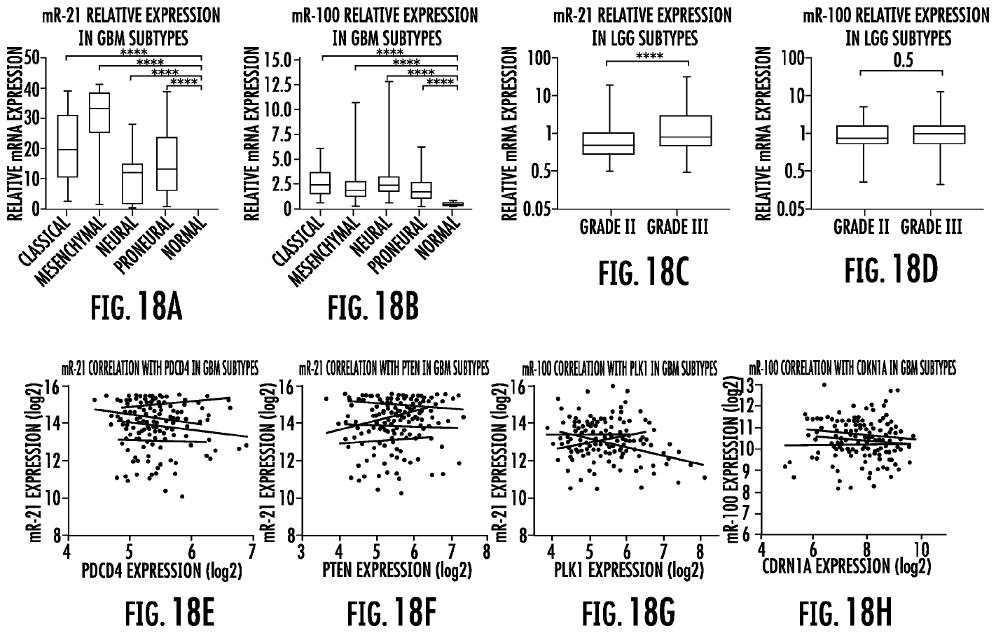

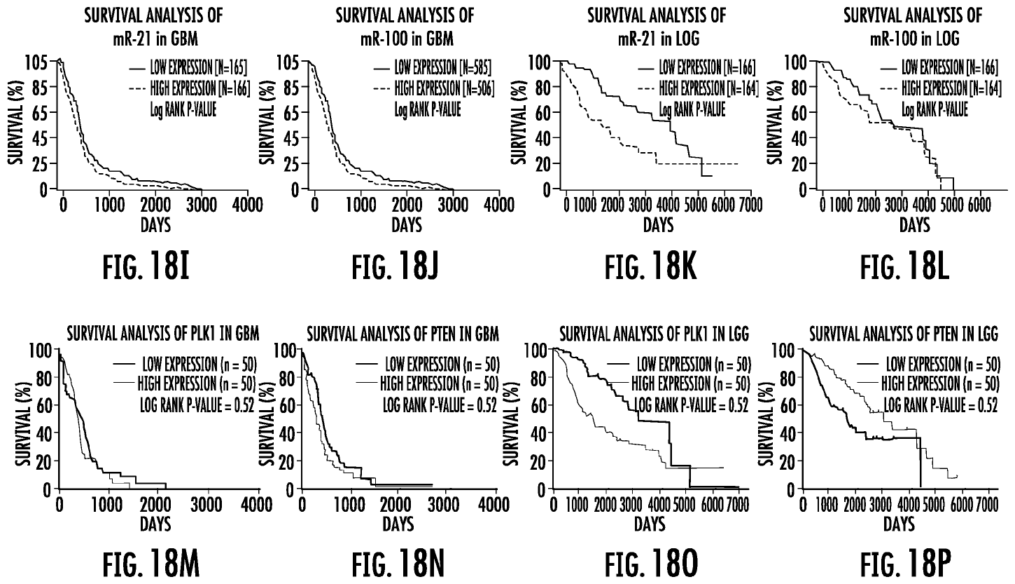

We also investigated the dynamics of nose-to-brain transport of mpEVs at different time points using in vivo and ex vivo imaging, and ex vivo quantitation of delivered miRNA using qRT-PCR in nude mice orthotopically implanted with U87MG GBM tumor (FIG. 17). The biodistribution study presented clear evidence of olfactory and trigeminal nerve branches-mediated transport of vesicles to the forebrains and brainstems of mice. Ex vivo quantitation of brain tissues segmented into three sections (forebrain, midbrain and hindbrain) indicated distinct migration patterns of mpEVs over the course of different periods (two animals were allocated for each time point). The ICG labeled mpEVs co-localized with the bioluminescence signal arose from the U87MG tumor masses at 24 h, which correlated well with the ex vivo miRNA quantitation results. It was evident from these results that during the initial timespan the olfactory nerves dominated as the route of entry into the forebrain, whereas at later time points, trigeminal nerve branches contribute to intracranial delivery toward the brainstem and hindbrain. RT-PCR results also correlated with mpEVs fluorescence distributions that we observed in ex vivo brain imaging. Likewise, whole head histological sections showed the presence of mpEVs in the olfactory mucosa (the olfactory neuroepithelium), which undergo paracellullar or transcellular transport to reach the olfactory receptor neurons. Upon intracranial arrival of mpEVs through the olfactory nerve pathways, they can be distributed via CSF and interstitial fluid within the brain to eventually exhibit CXCR4-SDF1 tropism towards GBM. On the other hand, the other major route of intracranial entry from the nose through trigeminal nerve branches of the respiratory epithelium was evident in histological head sections (FIG. 18). High resolution images of different sections of trigeminal nerves and their maxillary division branches at different anatomical locations presented clear evidence of ICG labeled EVs. The transport of ICG labeled mpEVs through the trigeminal nerves subsequently led to their accumulation in the hindbrain, including the pons, medulla, and spinal cord, which correlated well with ex vivo imaging and RT-PCR quantification of delivered miRNAs.

### Discussion

The results provided here demonstrate that mpEVs processed using a microfluidic system can efficiently load miRNAs using a dissociation and reassembly-based reconstitution process. (FIG. 2). Furthermore, NSC-mpEVs engineered to overexpress CXCR4 receptors can be loaded with anticancer miRNAs to enhance their intranasal delivery and subsequent trafficking to intracranial GBMs while bypassing the BBB. These EV therapeutic carriers can also have immunomodulatory effects on targeted tumors to improve treatment response. We chose anti-miRNA-21 to silence tumor-associated oncomiRNA-21 function, as well as miRNA-100 mimics to enhance p53-mediated tumor suppressor function in GBM cells having wildtype p53 expression, or to promote PLK1-mediated apoptotic induction in GBM cells having mutant p53 or p53-null genotypes.

Extracellular vesicles include exosomes 30-100 nm in size originating from inward budding of inner membranes and microvesicles of heterogeneous size (100-1000 nm) produced by outer membrane blebbing. Although biogenesis of both types of vesicles are produced by different mechanisms, both can load intracellular cargos and have different surface markers. In the work described above we used microvesicles isolated from engineered NSCs for processing into mpEV nanovesicles. The microfluidic-based self-assembly strategy for mpEV production overcomes many of the constraints associated with the conventional generation of extracellular vesicles. Specific advantages of the system described here include: (1) scalability, (2) transfection free loading of therapeutics, (3) freedom from synthetic agents for transfection, (4) the mpEVs are of uniform sizes and (5) uniform loading of microRNAs.

Most enteral and parenteral routes of therapeutics delivery rely on blood vasculature to reach GBMs. However, an intact BBB, and a variable blood-tumor barrier, hinder the delivery of many therapeutics to intracerebral GBMs. Recently, Sukumar et al. have shown that intranasally administered miRNA-loaded gold-iron oxide nanoparticles effectively delivered intact miRNAs to GBMs and functionally improved their response to TMZ. A major advantage of the intranasal delivery route is avoidance of serum degradation, as well as expected biological elimination of therapeutics by the liver, gastrointestinal tract, and kidneys. Upon intranasal administration, therapeutics may be transported across the nasal epithelium by intracellular and/or extracellular pathways. The extracellular route likely provides the pathway by which NSC-mpEVs could cross the nasal epithelium and then migrate along branches of the olfactory and trigeminal nerves. That, together with endocytosis into olfactory and trigeminal nerve branches and subsequent axonal transport may provide facilitated intracranial access to the cerebrospinal fluid and brain parenchyma. Here, we used tumor targeted NSC-mpEVs for intranasal administration to enhance delivery and targeting of therapeutic miRNAs to GBMs while bypassing the BBB.

The CXCR4-SDF1e axis is upregulated in various cancers, including GBMs, and the levels of CXCR4 receptors are low in background mature neurons. Hence, we hypothesized that if we overexpress CXCR4 in NSCs, this, in turn, could yield MVs and mpEVs with enriched CXCR4, which can further enhance targeting ability of CXCR4-NSC-mpEVs to GBMs secreting SDF1e. Here, we convincingly show that CXCR4-NSC-mpEVs improve the delivery of miRNA cargos to GBM cells in vitro.

Although we engineered NSCs to overexpress CXCR4, the control NSCs also inherently express basal levels of CXCR4. Thus, mpEVs derived from control NSCs with basal levels of CXCR4 would also be expected to reap marginal benefit of CXCR4-SDF-la tropism. Consequently, therapeutic effects of miRNA-loaded mpEVs were comparable to those of miRNA-loaded CXCR4 mpEVs in combination with TMZ. That said, and although the difference is not clearly evident in our in vitro results, we observed a significant increase in extent of miRNA delivery in CXCR4-mpEVs treated mice compared to that of control-mpEVs, which evidently manifested a prominent increase in mice survival and tumor regression. It is also crucial to consider that in the course of CXCR4-mpEVs transit from nose to brain, CXCR4 targeting comes into play only after EVs reach the intracranial space via the olfactory and trigeminal nerve branches, which is also consistent with the fate of control-mpEVs. Similarly, we demonstrate enhanced in vivo delivery to mouse GBM tumors upon intranasal delivery via the nose-to-brain pathways Importantly, this improved the response of GBMs to standard systemic TMZ therapy. We demonstrate the efficacy of this novel combination treatment in mice by size reduction of GBMs as determined on MRI and photoacoustic imaging, and the ensuing extended survival of animals. We believe this to be an important finding because, although TMZ is the current frontline chemotherapy for GBMs, its effectiveness alone is limited by its low potency as an alkylating agent, dose-limiting systemic toxicity, rapid degradation, and development of drug resistance.

Even though intranasal delivery can deliver therapeutic mpEVs to the brain, there was a relatively strong ICG fluorescence signal in the lungs, which was likely consequent to inhalation of NSC-mpEVs during intranasal administration. As observed by Sukumar et al., the respiratory rate of mice is an important factor in determining the intranasal residence time and subsequent migration of NSC-mpEVs. Slow breathing under deep anesthesia was found to improve localization of NSC-mpEVs to the nasal cavity, thus facilitating their migration into the brain via the nose-to-brain pathways. In contrast, under mild anesthesia accompanied by relatively fast breathing rates and short intranasal residence time, the mpEVs were not effectively absorbed in the nasal cavity and moved into lungs and digestive tract. Notably, however, we did not see any toxicity in lung tissues. This technicality aside, the adopted administration approach led to consistent patterns in ICG-labeled mpEVs distribution in the brains of mice characteristic of two established pathways via branches of the olfactory and trigeminal nerves present in the olfactory neuroepthelium and respiratory epithelium of the nasal cavity, respectively. The delivered mpEVs were significantly concentrated in the rostral and caudal regions of the brain, indicating that the nanoparticles administered intranasally had successfully trafficked along these two pathways.

We also performed in silico analysis of the therapeutic roles of miRNA-21 and miRNA-100 to correlate our results for these two chosen therapeutic miRNAs with potential future clinical translation. We found that these two miRNAs do indeed have significant roles in oncogenesis, and they correlate with survival in patients with GBM.

### Materials and Methods

Materials. Cell culture plates, FBS, penicillin, streptomycin, sodium bicarbonate, cell culture medium and phosphate-buffered saline (PBS) were purchased from GIBCO BRL (Frederick, MD). Lipofectamine 2000 transfection reagent was from Invitrogen (Carlsbad, CA). All other chemicals were purchased from Sigma-Aldrich. Cy5-labeled anti-miRNA-21 and miRNA-100RNA oligonucleotides were synthesized at the Protein and Nucleic Acid facility at Stanford University.

Cell culture. U87MG and GL26 glioma cells were cultured in Dulbecco's Modified Eagle's Medium supplemented with 10% FBS and 100 U/ml penicillin and 0.1% streptomycin in a 37° C. incubator with 5% C02 and 95% air. Cells were tested for mycoplasma contamination using a MycoAlert kit from Lonza (Allendale, NJ), and maintained them at optimum culture conditions as per ATCC guidelines.

### CXCR4 Receptor Engineering of Neural Stem Cells and Characterization by FACS Immunoblot, and Fluorescence Microscopy.

We evaluated the NSCs engineered to display surface CXCR4 receptors for this modification by directly measuring the expression levels of CXCR4 protein using immunoblot analysis, and indirectly by measuring ICG-fluorescence in NSCs using FACS and fluorescence microscopy analysis. For immunoblot analysis using target protein specific antibodies, we seeded 50,000 control cells and a similar number of CXCR4-NSCs in 12-well plates. After 48 h of seeding, we harvested cells and process them for further immunoblot analysis using anti-CXCR4 receptor antibody. In brief, we lysed cells in 100 ml lysis buffer, and mixed 80 μg of total protein from treated control NSCs and CXCR4-; >4S With a-mercaptoethanol (Bio-Rad) and 10 μl of NuPAGE LDS (4×) loading buffer, then heated at 90° C. for 5 min and in 4-12% SDS-polyacrylamide gel electrophoresis gradient gel (Invitrogen), and then ran this at 80 V for 2 h. The resolved proteins from the gel were then electroblotted onto a 0.2 μm pore-size nitrocellulose membrane (Schleicher & Schuell, Keene, NH, USA). The membrane was blocked with 5% non-fat dry milk in tris-buffered saline containing 0.01% Tween-20 (TBS-T, pH 7.6) for 30 min and incubated with the appropriate polyclonal antibody overnight at 4° C. on a rocking platform. On the following day, we replaced the primary antibody with peroxidase-conjugated goat anti-rabbit IgG secondary antibody (1:10,000 dilution, Rockland Immunochemicals, Gilbersville, PA, USA). After washing it thrice for 10 min in TBS-T buffer, and rocking for 2 h, we developed the blots using pierce ECL Western Blotting Substrate (Thermo Fisher Scientific, USA) and imaged and quantified them using an IVIS Lumina III in vitro imaging system. For FACS, we used 100 μl of control and CXCR4-NSCs at 4×10⁵ cells/ml. The Grava easyCyte Flow Cytometer Millipore was used for analysis. The optimal window for analysis was set by using PBS to eliminate debris, and control NSCs were used to set compensation and CXCR4-NSCs as positive samples. We collected 10,000 events from each sample for analysis. Similarly, for fluorescence microscopy, 5 μl of control and CXCR4-NSCs at 4×10⁵ cells/ml were placed on a glass slide, and a coverslip was placed without generating bubbles. The slides were imaged for CXCR4-NSCs ICG fluorescence using a Leica laser confocal microscope with a 40× oil immersion lens.

Transfection of Anti-miRNA-21 and miRNA-100 into Control NSCs and CXCR4-NSCs.

For the efficient loading of Cy5-anti-miRNA-21 and miRNA-100 into control NSCs and CXCR4 engineered NSCs, we plated these cells to 70% confluence (4×10⁶ cells per 10 cm plate) 24 h before transfection. After 24 h, the cells were washed once with PBS, and we added 8 ml of fresh medium. For transfection, 5 nmol of Cy5-anti-miR-21 and miR-100 was complexed with 25 ul of lipofectamine 2000 transfection reagent in 1 ml of serum-free optiMEM as per the manufacturer's instructions. After 45 min of incubation at room temperature in the dark, the complex was mixed well and added to the plates. After 16 h of transfection, the plates were washed once with PBS, and we added 8 ml of serum-free DMEM with penicillin and streptomycin for 24 h. The plates were kept in a shaker (300 rpm) for an additional 12 h for the release of EVs. The medium was collected and used for isolation of extracellular vesicles (EVs) as described later.

Collection of Microvesicles from Control NSCs and CXCR4-NSCs.

In brief, we cultured the control and CXCR4 engineered NSCs to attain 70% confluence in complete medium containing FBS. We then rinsed the plates twice with PBS before addition of serum free medium (SFM) and placed on a shaker (300 rpm) (model 260350, Boekel Scientific) for 12 h in an incubator at 37° C. and 5% C02. We collected the supernatant SFM in the plates and centrifuged this at 300 g for 15 min to remove cells and debris, and then centrifuged again at 2,000 g for 15 min to remove apoptotic bodies. Total EVs were collected by ultracentrifugation at 100,000 g for 4 h using a SureSpin rotor. The pellet was carefully suspended in the required volume of PBS and used for further characterization and studies.

Microfluidic Processing for Collected Microvesicles from Control NSCs and CXCR4-NSCs.

MVs collected after ultracentrifugation and processed by the microfluidic system are referred to as mpEVs or nanovesicles, due to their uniform size in the nanometer range. After collecting MVs from control and CXCR4-NSCs, we added ICG (200 μg, 5 mg/ml) into the MVs suspension, and then placed the suspension in a shaker for 2 h, followed by ultracentrifugation at 100,000 g for 4 h until the pellet labeled with ICG dye deposited at the bottom of the centrifuge tube. The pellet was carefully suspended in the required volume of upper suspension. We used 10 nmol (200 pmol/X) of each miRNA (Cy5-anti-miRNA-21 and miRNA-100) with 6.0×10¹⁰ MVs in 6 ml of physiological saline, and subjected each to the LV1-microfluidic system processing. We set the LV1-microfluidics system (Microfluidics, Westwood, MA) at 30,000 psi, and washed the system working-track with 75% ethanol solution twice, and then re-washed with PBS three to four times. We injected the MVs suspension into the microfluidic system and extracted the mpEVs at the outlet. Finally, we prepared three kinds of mpEVs: (1) control mpEVs (consisted of equal amounts of MVs from control and CXCR4-NSCs without loaded miRNAs); (2) control mpEVs plus miRNAs (mpEVs from control NSCs loaded with miRNAs); and (3) CXCR4-NSC-mpEVs plus miRNAs (mpEVs from CXCR4-NSCs loaded with miRNAs).

Comparison of mpEVs from Microfluidic System Processing and Transfected NSCs Using FACS and qRT-PCR Analysis.

We assessed the mpEVs obtained from microfluidics system processing and those NSCs transfected with Cy5-anti-miRNA-21 and miRNA-100 for miRNA loading efficiency by indirectly measuring Cy5-fluorescence of these vesicles using FACS, and by directly measuring the quantity of loaded miRNAs in vesicles using qRT-PCR. For FACS analysis, we used 100 μl of each type of vesicles at 1.0×10¹⁰ vesicles/ml, and the Guava easyCyte Flow Cytometer from Millipore for analysis. We then analyzed results for EVs based on their fluorescence intensities using FlowJo 10.6.1 software.

Quantitative Comparison of miRNA Loading in EVs from Transfected NSCs and Reconstructed mpEVs.

We used quantitative TaqMan RT-PCR to evaluate the miRNA loading efficiency of mpEVs and EVs. For this assay, we used equivalent numbers of vesicles before microfluidics to normalize miRNA encapsulation efficiency with respect to EV membrane composition. In brief, 10 nmols of miRNAs (10 nmols each of anti-miRNA-21 and miRNA-100) were complexed with 50 μl lipofectamine and transfected to NSC cells at 70% confluence in 10 cm plates. We washed the cells 6 h after transfection to remove extracellular lipofectamine and miRNAs. We then collected the vesicles from four such plates 24 h after transfection using ultracentrifugation, as described earlier. Likewise, we harvested control EVs from an equivalent number of NSC cells under similar culture conditions and used these for microfluidic-based reconstruction of vesicles (mpEVs) to load an equivalent amount of miRNAs (i.e. 10 nmols each of antimiRNA-21 and miRNA-100) FIG. 10. We used a similar batch prepared using Cy5-anti-miRNA-21 and miRNA-100 loaded EVs and mpEVs for FACS analysis.

Electron Microscopic Imaging of Processed mpEVs.

Field emission TEM images of microRNA loaded NSC-mpEVs processed by microfluidic system were acquired using JEOL JEM1400 that was operated at an acceleration voltage of 120 kV. Images were obtained using an Gatan OneView camera through Digital Micrograph interface. The samples were prepared by drop casting 10 μL of NSC-mpEVs suspension on carbon coated copper grids and incubated for 10-15 min. After the incubation period the samples were negatively stained with 1% uranyl acetate solution and incubated for 1 minute. The negatively stained samples were then briefly rinsed with ultrapure water to remove excess negative stain then air dried and mounted on the sample holder for imaging.

RNA Extraction and qRT-PCR Analysis.

We quantified the delivery of therapeutic miRNAs to cells in in vitro experiments and tissues from in vivo studies using TaqMan real-time qRT-PCR. We treated U87MG cells with control mpEVs and CXCR4-NSC-mpEVs loaded with miRNAs (anti-miRNA21 and miRNA100) plus TMZ, and at the end of 48 h we harvested the cells for analysis. Likewise, we used tumor tissue and organs harvested from each treatment group of the in vivo study to quantify EV delivery. The cells and tissues were lysed and homogenized in 300 μl of lysis buffer for 3 min. We supplemented the lysis buffer with 30 μl of homogenate additive and incubated at room temperature for 10 min. The RNA was extracted once with phenol and chloroform, and purified using a column, washed with wash buffer, and eluted in 50 μl of sterile distilled water. We quantified the total RNA and checked for purity using a Nanodrop spectrophotometer. After quantification, 50 ng of total RNA was reverse-transcribed using RT primers (Life Technologies) using a reverse-transcription kit (Life technologies) to produce corresponding cDNA. The cDNA synthesis was carried out in a 15 μl reaction volume. qRT-PCR was performed using cDNA (5 ng of RNA equivalent) combined with TaqMan-PCR reagents (primer and probe mix). qRT-PCR was performed by 2 min incubation at 50° C. followed by activation of the DNA polymerase at 95° C. for 10 min, 60 cycles of 95° C. for 15 s, and 60° C. for 60 s in an Eppendorf Real flex qRT-PCR system (Eppendorf AG, Hamburg, Germany). The qRT-PCR reaction was carried out in a 20 μl reaction volume. The expression of miRNAs was calculated using the ^{2-ΔΔ} method.

Sodium Dodecyl Sulfate-Polyacrylamide Gel Electrophoresis SDS-PAGE Analysis of Cy5-miRNA Loaded and ICG Labeled mpEVs.

We measured the protein concentrations of mpEVs using NanoDrop (Malvern Panalytical Ltd, Malvern, UK), and then 15 μg of protein was denatured in SDS sample buffer (Invitrogen, Carlsbad, CA). The samples were then resolved in a NuPAGE Novex 4-12% Bis-Tris 10-well mini gel in 3-(N-morpholino) propanesulfonic acid running buffer using a Novex-SureLock-Xcell Electrophoresis System (Invitrogen). We stained the gel in Coomassie Blue (Invitrogen) staining buffer for 1 h and further washed in water for de-staining. We then imaged Coomassie stained gels on a Gel Doc EZ system (Hercules, CA, USA) fitted with a white light conversion screen controlled through Image Lab 2.0.1 software. The presence of Cy5-miRNA and ICG labeled EV proteins in the same gel was assessed using an IVIS Lumina II (Caliper Life Sciences, Hopkinton, MA, USA) quantitative fluorescent imaging system with excitation at 710 nm for ICG and 640 nm for Cy5.

### Dynamic Light Scattering (DLS), Potential and Nanoparticle-Tracking Analysis (NTA).

We established the mean particle diameter (z average), surface charge (potential), and size distribution (polydispersity index) of mpEVs. For DLS measurements, the samples were diluted in 18 MS2 water, and data from at least three measurements were averaged. The potential was measured at pH 7.4. The Smoluchowski approximation was used to convert the electrophoretic mobility to a potential. For NTA analysis, we further diluted mpEVs with and without loaded miRNAs 50-fold to 500-fold for the measurement of particle size and concentration in media. For each sample, at least five videos of 30-60 s duration, with more than 200 detected tracks per video, and in at least one dilution, were taken and analyzed using the NTA software with the default settings.

Analysis of Cellular Uptake of Cy5-Anti-miRNA-21 Loaded and ICG Labeled mpEVs.

Initially, we investigated GL26 glioma cell uptake efficiency of mpEVs using control and CXCR4-NSCs, with and without loaded miRNAs, all with concomitant TMZ treatment. For this purpose, we incubated GL26 glioma cells (1×10⁵ cells per well) plated in 6-well plates with 6 μl mpEVs from control and CXCR4-NSCs loaded with Cy5-anti-miRNA-21 and labeled with ICG for 48 h at 37° C. and 5% C02, along with the TMZ treatment at a dose of 100 μM. After incubation, we fixed the cells with 4% paraformaldehyde in PBS for 10 min, washed in PBS, permeabilized with Triton-X, and stained with 3 μM Hoechst 33258 for 5 min, and then imaged with a Leica fluorescence microscope. We also evaluated the time- and TMZ concentration-dependent glioma cell uptake of mpEVs using FACS analysis. Similarly, we incubated U87 glioma cells (1×10⁵ cells per well) plated in 24-well plates with 2 μl mpEVs from control and CXCR4-NSCs with or without loaded Cy5-anti-miRNA-21 for 24 h and 48 h at 37° C. and 5% C02. For TMZ concentration-dependent uptake, we treated the U87MG glioma cells with different TMZ concentrations (250 μM and 500 μM). After incubation, we collected the cells and fixed them for subsequent FACS analysis, as described earlier.

### MicroRNA Loaded EV Uptake in 3D-Spheroid Cell Culture of U87MG Cells.

We established 3D spheroid cell cultures of 100,000 U87MG cells in 100 microliters of Matrigel® matrix on a cover slip within a 6-well plate, and incubated for 24 h. We then treated these U87MG organoids culture with Cy5-labeled miRNA (200 pmol anti-miRNA-21, and 200 pmol miRNA-100) loaded control mpEVs or CXCR4-mpEVs, and incubated for 48 h. We washed the spheroids with 0.2 ml of 1×PBS and fixed with 0.2 ml of 4% paraformaldehyde for 10 min, and followed by treatment with 0.2 mL of 4 μl/mL DAPI in PBS for 10 min at room temperature for nuclear staining. Finally, we washed the spheroids in 0.2 ml PBS and once in 0.2 ml deionized water. Spheroids were then mounted with ProLong Antifade Mountant media on a glass slides and imaged using confocal microscopy (TCS SP8, Leica). We acquired the spheroid images at different sections under DAPI, Cy5 and ICG filters to identify the cell nuclei, Cy5 miRNAs (anti-miRNA-21 and miR-100) and ICG labeled mpEVs, respectively. We overlayed and analyzed the acquired images to estimate the depth of EV penetration and miRNA distribution using LAS X software.

Glioma Cell Viability and Chemosensitizing Effect of miRNAs-Loaded mpEVs Using FACS Analysis.

To investigate the chemosensitizing effect of miRNAs-loaded mpEVs in the absence and presence of TMZ at different concentrations, we seeded the U87 glioma cells at a density of 1×10⁵ cells per well in 12-well plates. Next, we treated the cells with mpEVs from control and CXCR4-NSCs with and without loaded miRNAs. On the following day, we treated the cells with TMZ at different concentrations (0 μM, 250 μM, and 500 μM) for each kind of EV. At 24 h after TMZ administration, we collected the cells and fixed in ice cold 70% ethanol at −20° C. for 30 min. We then washed the fixed cells once with 2 ml of PBS and stained with PI (15 nM) in the presence of RNase A (10 mg/ml) and 0.01% Triton-X100 (Life Technologies, Grand Island, NY, USA). We performed FACS analysis for each treatment condition and displayed the results as histograms with percentages of live and apoptotic cells. Likewise, the chemosensitizing effect of miRNA loaded mpEVs for TMZ therapy was also investigated against GL26 cells by using a similar procedure.

### Stereotaxic Intracerebral Implantation of Glioma Cells in Nude Mice and C57/BL6J.

After initiating isoflurane gas anesthesia (2%) in oxygen to maintain a slow and regular respiratory rate, we placed each nude mouse (6 weeks, 23.0-25.8 g body weight) on the stereotactic frame with the head fixed. Under sterile conditions, we created a burr hole at standard coordinates between bregma and lambda using a small drill (1 mm diameter). We inserted the needle tip into mouse brain gently to a depth of 5 mm to then slowly inject 5×10⁵ GFP-labeled U87MG glioma cells suspended in 5 μl PBS. After slow needle removal, we sealed the burr hole with bone wax, sterilized the inoculation site, and sutured the overlying scalp. Likewise, we also validated the outcome of the study in GL26 orthotopic brain tumor model in immunocompetent C57/BL6J mice and evaluated therapeutic outcomes of equivalent treatment groups. The miRNA-loaded mpEVs were delivered intranasally in mice. The miRNA delivery and TMZ administration were followed as indicated in FIG. 6A.

In Vivo Intranasal Administration of miRNAs Using mpEVs.

We performed all animal experiments under the guidance of the Administrative Panel on Laboratory Animal Care (APLAC), Stanford University. We obtained all nude mice (Nu/Nu) and C57BL-6J mice from Charles River laboratories (Wilmington, MA) and Jackson Laboratory (Bar Harbor, ME). We performed the intranasal delivery of mpEV formulations in mice under partial anesthesia using isoflurane gas in Oxygen, to enable animal recovery within a couple of minutes. We placed each animal in the induction chamber and adjusted the oxygen flowmeter to 0.8 to 1.5 L/minute. Once completely anesthetized, the animals were positioned in a 60° angle in the supine position and monitored for a steady breathing rate. We then administered 20 μL of the miRNA-loaded mpEVs (6×10¹² mpEVs loaded with 4×10¹⁵ copies of miRNA) as 5 μL/drop slowly into the nares over 15-20 min. After administering each drop, we stopped for 3-4 min to ensure the animal inhaled the drop and that it was breathing at a steady rate. The nostrils were observed closely for signs of blockage or irritation. After administering the full dose, each animal was allowed to recover from anesthesia before transferring to its cage.

### Fluorescence and Bioluminescence Imaging.

To acquire ICG-fluorescence images (at 745/830 nm) and tumor bioluminescence at predetermined time points of the study, we first anesthetized the mice through a nose cone using 2% isoflurane in oxygen. We then imaged the mice using a Lago spectral instruments imaging system (Tucson, AZ). After one week of intracranial glioma cell implantation, the tumor bioluminescence was considered as the baseline and then mice were randomized into treatment groups. We administered D-luciferin and TMZ intraperitonially, and mpEVs intranasally at specific time points as indicated in the schematic (FIG. 6A). At every time point, we bioluminescence imaged the tumors upon intraperitoneal administration of the substrate D-Luciferin (3 mg). We administered 20 μl of EV suspension to mice every other day. Specifically, successive aliquots of 5 μl EV suspension were slowly delivered under deep anesthesia using isoflurane gas (2%) in oxygen. We obtained fluorescence imaging every day, with or without intranasal administration of mpEVs. We also recorded the mouse weight at each time point and thereafter for 48 days. We sacrificed all animals on Day 48, and we collected the organs and tumors for ex vivo quantification of miRNA delivery and histological analysis. For anti-mRNA-21 and miRNA-100 biodistribution in different organs, we used TaqMan real-time qRT-PCR, as described earlier.

MR imaging.

We imaged each implanted GBM using a 3T MRI scanner for small animals (MR Solutions, Guildford UK) at day 12, after anesthesia with isoflurane gas (2%) in oxygen. Standard T1- and T2-weighted imaging, and gadopentetate meglumine contrast-enhanced T1-weighted imaging were performed using a mouse body coil (MR Solutions Ltd.), and the images were transferred to a local workstation and analyzed using commercial 3D analysis software (OsiriX, Pixmeo SARL, Switzerland).

### Ex Vivo Photoacoustic Imaging of IGC Labeled EV Migration in Brain.

The accumulation of ICG labeled mpEVs into the brain was assessed using the Endra Nexus 128 photoacoustic imaging system (Endra Inc., Ann Arbor, MI). To avoid background photoacoustic signal generated by impedance mismatch between the skull and underlying brain tissues, the intact brain was harvested from each treatment group and then scanned immediately using a hemispherical photoacoustic imaging system. The system uses a tunable, near-infrared Nd:YAG laser that produces 7 ns pulses at a fluence of 2.5 mJ cm⁻² with a pulse repetition rate of 20 Hz. The wavelength could be tuned within a range of 680-980 nm. At the laser repetition rate of 20 Hz, a standard scan using 1800 bowl positions took 15 min per excitation wavelength. We acquired photoacoustic images of the brain (180 angles, 20 pulses/angle) with laser light at 710 nm and 900 nm (24 s total scan time, 240 views, 1 pulse/view). Ultrasound detection was achieved through a hemispherical ultrasonic device that consists of 128 ultrasonic transducers. The data were analyzed and images were reconstructed using Osirix software (Apple, Cupertino, CA).

### Hematoxylin and Eosin Staining.

At the termination of all in vivo imaging studies, we sacrificed each mouse using standard procedures, and fixed harvested tissues in 4% paraformaldehyde overnight at 4° C., immersed them in 70% ethanol, embedded them in paraffin, and then sliced at 5 μm thickness using a Leica cryo-microtome (Leica RM2255). We stained these sections in undiluted hematoxylin (Sigma-Aldrich, USA) for 2 min, rinsed in running water, and differentiated in 1% HCl acid/alcohol for 30 s. We then washed these slices and immersed them in bluing solution (Fisher, USA) for 1 min, washed in running water and rinsed in 10 dips of 95% alcohol. We then counterstained the slides in eosin by dipping into 1:5 ethanol diluted eosin solution (Fisher) for less than 30 s, dehydrated through 95% alcohol, and then xylene for 5 min each. We mounted slides with xylene based mounting medium (Permount, Sigma-Aldrich, USA) and imaged using Nanozoomer (Hamamatsu, Japan).

### Bone Decalcification and H&E Staining of Mice Heads.

At the conclusion of the study and after animal sacrifice, the mice heads were harvested intact, de-skinned and immersed in mild decalcifying solution Cal Ex II (composed of formic acid [decalcifying agent] and formalin [fixing agent]) for 72 h. We then transferred decalcified skulls to 70% ethanol, embedded in paraffin and used for sectioning. The paraffin embedded heads were sectioned into slices 5 μm thick. The processed slices were then used for H&E staining.

### Tile Scanning of Decalcified Mice Heads Using Confocal Microscopy.

The paraformaldehyde fixed tissues were embedded in paraffin and sectioned in 5 μm thick slices. The slides were deparaffinized in xylene and rehydrated by gradient alcohol washes, then washed three times for 15 min each in wash buffer. We stained the tissues for actin using 5 μl solution of Alexa Fluor 488® phalloidin (66 μM) diluted in 200 μl of 1% bovine serum albumin (BSA) in PBS. The tissue slices were incubated in staining solution for 20 min at room temperature and rinsed twice with PBS. We subsequently stained the cell nucleus by supplementing 300 μl of the diluted Hoechst 33258 solution on tissue slices followed by incubation at room temperature for 5 min. We then rinsed the slides twice with PBS and mounted with Permafluor anti-fade mounting media. We imaged the slides using confocal microscopy (TCS SP8, Leica). Optical sections were acquired in all three channels along x-y planes obtained at different intervals (z step=6) along the optical axis (10-30 μm). Images were presented as multichannel, and maximum projections were generated over the whole stack after deconvolution. The tile scanned sections of each slide were mosaic merged, stitched and reconstructed using LAS-X software reconstruction.

### In Silico Analysis.

We verified the expression of miRNA-21 and miRNA-100, and the survival analysis for GBM and low-grade glioma (LGG) in The Cancer Genomic Atlas (TCGA) database. We obtained clinical information of patients and processed miRNAs expression data for GBMs from Project Betastasis (http://www.betastasis.com/), which included 196 GBMs and 10 samples from adjacent normal brain tissue. The microarray data was analyzed based on the Agilent Human 8x15K miRNA platform. We categorized the GBM samples into four different subtypes, including classical (n=52), mesenchymal (n=58), neural (n=30) and proneural (n=56). The miRNA expressions from TCGA miRNA sequence data for LGG were obtained from OncoLnc (http://www.oncolnc.org), which included 506 patients. We stratified LGG patients into 298 grade II, including astrocytomas, oligodendrogliomas and mixed gliomas, and 208 grade III, including neoplastic astrocytomas and neoplastic oligodendrogliomas, based on their diagnosis in TCGA. We obtained clinical information for those patients who had survival information.

To test the expressional differences for each miRNA, first, we calculated the mean value for the normal tissue samples for each miRNA and then we divided all the samples for that miRNA by this value to come up with the relative expression of GBM vs. normal. A similar normalization method was used for grade II and grade III LGGs except that, because of a lack of normal brain tissue for this category of patients, the data were normalized to grade II expressions. Then we used a student t-test to compare GBMs vs. normal brain samples, and grade II vs. grade III gliomas. Moreover, the expression differences were tested between the normalized expression of each miRNA in different subtypes of GBM and normal brain tissue using a student t-test.

Correlations between each miRNA expression and downstream genes in GBM were observed by plotting the expression of miRNA and the genes. To better understand the role of miRNA on downstream signaling, we categorized patients into the four aforementioned subtypes and plotted each correlation separately. Linear regression lines were fitted to the data to show the slope. A negative slope meant that the miRNA might have a role in regulating the gene, but a positive slope meant that the miRNA does not have a regulatory role on that gene.

To find an association between the expressions of miRNA-21 and miRNA-100 and patient overall survival, we performed survival analysis. We divided LGG and GBM patients into high-expression and low-expression groups based on the top and bottom 33%. Overall survival curves were plotted using the Kaplan-Meier method to measure the survival association with gene expression and compared by log-rank test. The association between important therapeutic genes including PLK1 and PTEN and overall survival were also investigated in GBM and LGG patients using the same type of analysis as for miRNA.

Statistical analysis. All statistical analyses were performed using Prism 8.4.1 (GraphPad Software, LLC). The expression value of miRNA-21 and miRNA-100 in LGG and GBM were collected for each case and presented as mean±standard deviation. All statistical tests were two-tailed. The results were considered statistically significant when the corresponding P-value was less than 0.05.

### Supporting Methods and Results

### Methods

Stability and release profile of miRNA-loaded ICG labeled mpEVs: In the procedure used for synthesis of ICG-labeled mpEVs loaded with miRNAs, we covalently conjugated NHS-ICG to EV membranes. Upon completion of conjugation, the reaction mixture was subjected to high-speed ultracentrifugation to eliminate excess free ICG dye as well as free miRNAs. Thus, considering the additional washing step, we would not expect to see prominent leakage of ICG or free miRNAs from mpEVs. However, to further verify this claim and to check the stability of EVs and release of miRNAs from the final preparation, we investigated their release profile from the vesicles in a time dependent manner by dialysis under physiological conditions. We measured the released of miRNA by using TaqMan quantitative RT-PCR, and ICG by using fluorescence IVIS imaging. As the nose-to-brain transport occurs in a time span within 48 h, we restricted our release study to 48 h with appropriate time points to correlate with in vivo biodistribution results. We quantified the total amount of loaded miRNA in mpEVs and the released miRNA using TaqMan RT-PCR. We denoted the measured miRNA at each time point in terms of percentage total miRNA loaded in mpEVs.

### Results

Evaluation of miRNA distribution in m EVs using an RNase protection assay: To investigate the presence of miRNAs on the surface of mpEVs and to understand their contribution to the surface charge of the miRNA loaded mpEVs, we determined the zeta potential of miRNA-loaded mpEVs, with and without RNase treatments. We used increasing concentrations of RNase (0.5 μg/mL, 1 μg/mL, 1.5 μg/mL, 2 μg/mL, and 2.5 μg/mL) for 10 min, as well as increasing incubation times (0 min, 1 min, 2 min, 10 min, 20 min, and 30 min) at 1 μg/mL of RNase to perform an RNase protection assay. As indicated in FIG. 13, the zeta potential of RNase treated miRNA-loaded mpEVs increased gradually from −32 mV to −19.6 mV at 10 min of incubation with RNase. The increase in surface potential of miRNA-loaded mpEVs upon RNase treatment indicated that surface bound miRNAs were digested by RNase enzyme and led to increase in surface potential. However, at higher incubation times, i.e., 20 min and 30 min, we did not observe further increases in zeta potential, which indicated that the surface bound miRNAs were completely digested at this point, and supporting the fact that the remaining miRNAs were loaded within the mpEVs. The observed differences in surface potential of mpEVs in the absence of RNase (−24 mV), compared to its surface potential in the presence of RNase (−18.4 mV) at 10 min of incubation with 1 μg/mL RNase accounts for the degradation of surface bound miRNAs by RNase. Thus, to estimate the remaining miRNAs present within these vesicles (i.e., those inaccessible to RNase activity), we stopped the RNase activity at the end point of the incubation period by suspending in RNAzol, and then extracted miRNAs from mpEVs by acid phenol extraction followed by column purification. We quantified the remaining anti-miRNA-21 in mpEVs at each time points, i.e., 1, 2, 10, 20, 30 min by using TaqMan RT-PCR analysis. We observed a clear correlation between an increasing trend of surface potential of miRNA-loaded mpEVs with intact miRNAs extracted from respective treatment conditions. With increases in incubation periods, surface potential of miRNA-loaded mpEVs increased while the copies of anti-miRNA-21 loaded decreased for up to 10 min of incubation time. However, at later time points, as observed in the case of the zeta potentials, we observed no significant drop in anti-miRNA-21 copies, indicating that all the intact miRNAs remaining at those time points were loaded within the vesicles, thus rendering them inaccessible to RNase-mediated degradation. Quantification of miRNAs indicated the presence of 1.49×10⁸-fold anti-miRNA-21 in mpEVs treated with RNase for 10 min, whereas mpEVs without RNase treatment carried 2.1×10⁸-fold miRNAs. Comparison of these two ratios indicated that nearly 71% of miRNAs loaded in mpEVs were present within the vesicles. Thus, the RNase protection assay to measure surface zeta potential, and the TaqMan RT-PCR assay indicated the distribution of miRNAs in mpEVs when prepared by the microfluidic-mediated reconstruction process.

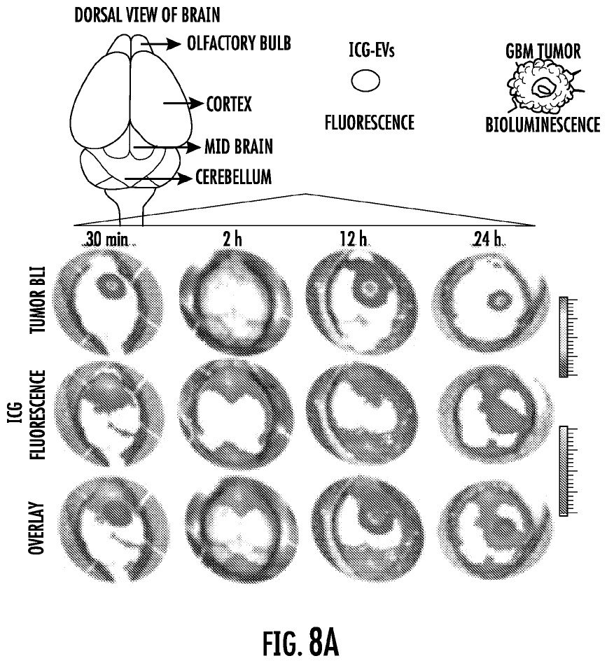

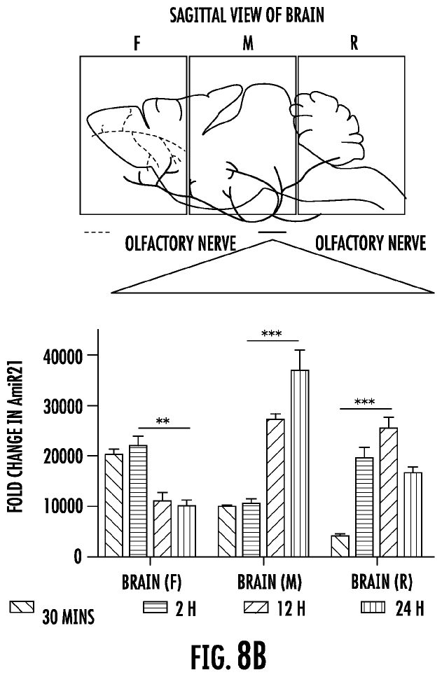

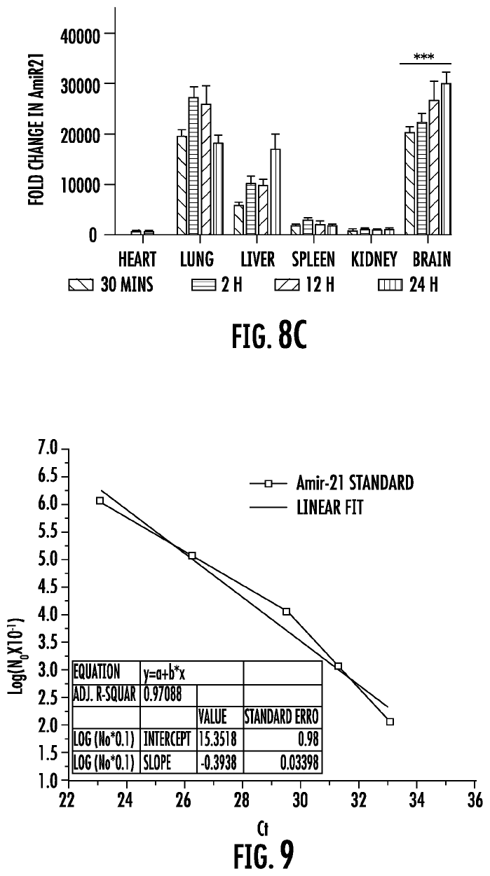

Stability and release profile of microRNAs and ICGs from mpEVs. The miRNA release profile from mpEVs exhibited an initial burst release of 7.42% upon incubation in physiological saline for 10 min and gradually increased and plateaued at later time points. At the end of 24 h, nearly 8% of loaded miRNA was released, and this did not change significantly until 48 h, indicating the stability of loaded miRNAs in mpEVs (FIG. 12A). Thus, considering the outcome of dynamic evaluations in nose-to-brain transport shown in FIG. 8B & FIG. 17, the time span for intranasally administered materials reaching the target brain region is relatively short (˜4 h) and the miRNA release profile at this time point is only around 8% of the total payload in mpEVs. Thus, most (about 92%) of miRNAs loaded in mpEVs remain intact within the vesicles at physiological conditions during the relevant time span of nose-to-brain transport, and thus available for further functional actions at target tissues. Additionally, we verified the stability of ICG conjugation to EVs by studying time dependent release of ICG using the Slide-A-Lyzer™ MINI Dialysis Device and subsequent quantification using the IVIS imaging system (FIG. 12B). The cumulative release of ICG until 48 h was estimated to be merely 3.51% of the total conjugated ICG, which indicated stability of ICG conjugated to EVs over the relevant time spans of nose-to-brain transport.

Histological analysis of NSCEV trafficking to brain after intranasal-delivery. We used control animals without intracranial tumors, with and without treatments (3 doses of CXCR4-NSC-mpEVs labeled with ICG over a week) for histological analysis of NSCEV trafficking to brain after intranasal deliver. We observed ICG fluorescence signal in the nasal mucosal epithelial cells and the submucosal soft tissues, confirming the uptake of ICG-labeled NSC-mpEVs by the nasal mucosal cells. Furthermore, the outer edge of the forebrain also revealed ICG fluorescence signal, and this extended deep into the brain. We did not observe any background fluorescence signal in control animals. Our findings again confirm the feasibility of intranasal administration of NSC-mpEVs, and their subsequent nose-to-brain migration to the site of a GBM in the brain. To further study the delivery in mice bearing GBMs and treated with TMZ in combination with NSCEV-miRNAs, we used four animal groups: (1) Control-NSCEV-ICG-miRNA; (2) Control-NSCEV-ICG-miRNA plus TMZ; (3) CXCR4-NSCEV-ICG-miRNA; and (4) CXCR4-NSCEV-ICG-miRNA plus TMZ. The cranial tissues collected after the study endpoint were used for multi-color confocal microscopy imaging. We observed clear delivery of CXCR4-NSCEV-miRNAs, and the consequent therapeutic effects upon co-treatment using TMZ.

In silico analysis We showed that the combination of miRNA-100 and anti-miRNA-21 significantly improved TMZ chemotherapy for GBMs in mice. Hence, we performed an in silico analysis of the expression of miRNA-21 and miRNA-100 from clinical tumor samples to correlate with the outcome of our results. We performed survival analysis for GBM and low grade gliomas (LGG) using The Cancer Genomic Atlas (TCGA) database. The clinical information of patients and the processed miRNAs expression data for their GBMs from 196 patients and 10 samples from the adjacent normal brain tissues were used for analysis. The results clearly showed a significantly higher level of expression of miRNA-21 in all subtypes of GBMs, including LGGs, compared to normal tissues (P<0.001). Similarly, miRNA-100 showed higher expression in GBMs and LGGs compared to normal, but the level was significantly downregulated in GBMs when compared to LGGs (FIG. 19A-FIG. 19D). We further analyzed expression levels of downstream targets of miRNA-21 (PTEN and PDCD4) and miRNA-100 (PLK1), including subtypes (FIG. 19E-FIG. 19H). The survival analysis of patients with the expression of miRNA-21 and miRNA-100 showed no correlation within GBM (FIG. 19I-FIG. 19P). In contrast, the expression of both miRNAs showed significant levels of variation within LGGs. Survival analysis of target genes expression with the tumor type showed significant correlation for PTEN and PLK1.

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All references, e.g., U.S. patents, U.S. patent application publications, PCT patent applications designating the U.S., published foreign patents and patent applications cited herein are incorporated herein by reference in their entireties. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

While this invention has been particularly shown and described with references to preferred embodiments thereof, it will be understood by those skilled in the art that various changes in form and details may be made therein without departing from the scope of the invention encompassed by the appended claims.

1. A composition comprising a plurality of nanovesicles encapsulating one or more therapeutic agents or imaging agents, wherein the nanovesicles have a mean particle diameter of from about 90-150 nanometers, from about 100-200 nanometers, from about 200-300 nanometers, from about 300-400 nanometers, or from about 400-500 nanometers, and wherein the nanovesicles comprise cell membranes of a source cell.

2. The composition of claim 1, wherein the cell membranes of the nanovesicles comprise a recombinant membrane associated protein, antibody, affibody, or peptide on the external surface of the nanovesicles.

3. The composition of claim 2, wherein the recombinant membrane associated protein binds to a ligand on the surface of a target cell, optionally wherein the recombinant membrane associated protein is a CXCR4 receptor.

4. The composition of claim 2, wherein the antibody is selected from an anti-transferrin receptor antibody, an anti-uPAR antibody, an anti-HER2 antibody, or a B7-H3 antibody.

5. The composition of claim 2, wherein the affibody is selected from an anti-transferrin receptor affibody, an anti-uPAR affibody, an anti-HER2-affibody, or a B7-H3 affibody.

6. The composition of claim 2, wherein the peptide is selected is from a T7-peptide, a urokinase plasminogen activator (uPA) peptide, or an SP94 peptide.

7. The composition of claim 1, wherein the one or more therapeutic agents is a nucleic acid, a small molecule therapeutic agent, or a mixture thereof.

8. The composition of claim 7, wherein the nucleic acid is an RNA or DNA, optionally wherein the RNA is a small interfering RNA (siRNA) or a micro-RNA (miRNA).

9. The composition of claim 7, wherein the nanovesicles comprise from about 500-800 copies of the nucleic acid, or a mixture of two or more nucleic acids.

10. The composition of claim 7, wherein the one or more therapeutic agents is a small molecule selected from one or more of doxorubicin, dexamethasone, gemcitabine, paclitaxel, sorafenib, tamoxifen, 4-hydroxy tamoxifen, or temozolomide.

11. The composition of claim 1, wherein the source cell is selected from the group consisting of stem cells, platelets, erythrocytes, lymphocytes, dendritic cells, monocytes, and tumor cells, optionally wherein the stem cells are neural stem cells and further optionally wherein the tumor cells are breast cancer cells.

12. The composition of claim 11, wherein the nanovesicles further encapsulate polymer-based nanoparticles containing the one or more therapeutic agents or imaging agents.

13. The composition of claim 1, wherein the polymer is poly(lactic-co-glycolic acid) (PLGA).

14. A method for loading one or more therapeutic agents into a plurality of cell-derived microvesicles, the method comprising subjecting a mixture of the one or more therapeutic agents and microvesicles to microfluidic processing to produce loaded microfluidic processed extracellular vesicles (mpEVs) having a mean particle diameter of from about 90-150 nanometers, optionally wherein the method further comprises subjecting the mpEVs to ultracentrifugation.

15. The method of claim 14, wherein the microfluidic processing comprises subjecting the mixture to a pressure of about 20,000-40,000 psi.

16. The method of claim 14, wherein the microvesicles are obtained from cells expressing a recombinant membrane associated protein, or functionalized using antibody, affibody, or peptide, optionally wherein the membrane associated protein is characterized in that it binds to a ligand on the surface of a target cell.

17. A plurality of nanovesicles loaded with one or more therapeutic agents produced by the method of claim 14.

18. A method of treating a disease or disorder, the method comprising administering to a subject in need of such treatment a composition according to claim 1, wherein the one or more therapeutic agents is indicated for treatment of the disease or disorder.

19. The method of claim 18, wherein the disease or disorder is a cancer, optionally a glioblastoma, a breast cancer, a hepatocellular carcinoma, or a lung cancer.

20. The method of claim 19, wherein the cancer is glioblastoma and the one or more therapeutic agents is a microRNA or a small molecule therapeutic agent selected from one or more of temozolomide, doxorubicin, or dexamethasone.

21. The method of claim 20, wherein the microRNA is an anti-miRNA-21, an miRNA-100, or a mixture thereof, optionally wherein the nanovesicles comprise from about 500-800 copies of the microRNA.

22. The method of claim 20, wherein the nanovesicles comprise a recombinant CXCR4 receptor on the external surface of the nanovesicles.

23. The method of claim 20, wherein the method further comprises administering temozolomide, doxorubicin, dexamethasone, or any combination thereof, to the subject.

24. The method of claim 19, wherein the cancer is breast cancer and the one or more therapeutic agents is a nucleic acid or a small molecule therapeutic agent selected from one or more of tamoxifen, 4-hydroxy tamoxifen, paclitaxel, and doxorubicin.

25. The method of claim 24, wherein the nanovesicles comprise a recombinant membrane associated protein, antibody, affibody, or peptide on the external surface of the nanovesicles.

26. The method of claim 25, wherein the antibody is an anti-uPAR antibody, an anti-HER2 antibody, or a B7-H3 antibody.

27. The method of claim 25, wherein the affibody is an anti-uPAR affibody, an anti-HER2-affibody, or a B7-H3 affibody.

28. The method of claim 25, wherein the peptide is a urokinase plasminogen activator (uPA) peptide.

29. The method of claim 24 wherein the method further comprises administering tamoxifen, 4-hydroxy tamoxifen, paclitaxel, doxorubicin, or any combination thereof, to the subject.

30. The method of claim 19, wherein the cancer is hepatocellular carcinoma and the nanovesicles comprise a peptide on the external surface of the nanovesicles, optionally wherein the peptide is an SP94 peptide.

31. The method of claim 30, wherein the one or more therapeutic agents is sorafenib, gemcitabine, or doxorubicin.

32. The method of claim 30, wherein the method further comprises administering sorafenib, gemcitabine, doxorubicin, or any combination thereof to the subject.

33. A composition comprising a plurality of nanovesicles, each comprising one or more therapeutic agents, wherein the nanovesicles have a mean particle diameter of from about 90-150 nanometers and wherein the nanovesicles comprise a coating.

34. The composition of claim 33, wherein the coating is a metallic coating, optionally wherein the metallic coating is gold, iron, or silver.

35. The composition of claim 33, wherein the coating is a polymeric coating, optionally wherein the polymeric coating is PLGA, PLGA-PEG, or chitosan.

36. The composition of claim 33, wherein the nanovesicles are obtained from dendritic cell membranes.
